(19) United States
(12) Patent Application Publication
(10) Pub. No.: US 2025/0018030 A1
(43) Pub. Date: Jan. 16, 2025

(54) PHARMACEUTICAL COMPOSITION FOR PROMOTING CANCER IMMUNOTHERAPY AND METHOD FOR PROMOTING CANCER IMMUNOTHERAPY

(71) Applicant: China Medical University, Taichung City (TW)

(72) Inventors: Hung-Rong Yen, Taichung City (TW); Chuan-Teng Liu, Gongguan Township (TW); Ying-Chyi Song, Taichung City (TW); Heng-Hsiung Wu, Taichung City (TW); Chun-Chieh Yeh, Taichung City (TW)

(73) Assignee: China Medical University, Taichung City (TW)

(21) Appl. No.: 18/650,689

(22) Filed: Apr. 30, 2024

Related U.S. Application Data
(60) Provisional application No. 63/526,790, filed on Jul. 14, 2023.

(51) Int. Cl. A61K 39/395; A61K 31/352; A61K 36/744; A61P 35/00; C12N 15/113

(52) U.S. Cl. CPC A61K 39/3955; A61K 31/352; A61K 36/744; A61P 35/00; C12N 15/113; C12N 15/1136; C12N 2310/14

(57) ABSTRACT

A pharmaceutical composition for promoting a cancer immunotherapy and a method for promoting a cancer immunotherapy are provided. The pharmaceutical composition includes an uncoupling protein 2 inhibitor and an interleukin-17 blockade. The method includes administering an effective concentration of a pharmaceutical composition to a subject in need thereof, in which the pharmaceutical composition includes an uncoupling protein 2 inhibitor and an interleukin-17 blockade.

### RELATED APPLICATIONS

This application claims priority to U.S. Provisional Application Ser. No. 63/526,790, filed Jul. 14, 2023, which is herein incorporated by reference in its entirety.

### BACKGROUND

### Technical Field

The present disclosure relates to a pharmaceutical composition and a method for promoting a cancer immunotherapy. More particularly, the present disclosure relates to a pharmaceutical composition for promoting a cancer immunotherapy and a method for promoting a cancer immunotherapy used thereof.

### Description of Related Art

Cancer, also known as malignancy, is a state of abnormal proliferation of cells, and these proliferating cells may invade other parts of the body as a disease caused by a malfunction in the control of cell division and proliferation. The number of people suffering from cancer worldwide has a growing trend. Cancer is one of the top ten causes of death for twenty-seven consecutive years.

Pancreatic ductal adenocarcinoma (PDAC) remains a largely incurable disease, owing to its frequent diagnosis in advanced disease stages and limited efficacy of systemic chemotherapy options, partly owing to robust desmoplastic tumor microenvironment (TME) that promotes immunosuppression and chemoresistance. Cancer immunotherapy has emerged as a promising therapeutic option in PDAC and its efficacy is maximized when stimulation of immune effectors is accompanied by elimination of immunosuppressive environment largely induced by TME.

Despite the recent successes of immune checkpoint blockade, the cancer immunotherapy failures remain common and are often accompanied by development of effector T cell metabolic insufficiency. Thus, metabolic targeting has emerged as a promising concept to augment the efficacy of the cancer immunotherapy.

### SUMMARY

One embodiment of the present disclosure provides a pharmaceutical composition for promoting a cancer immunotherapy. The pharmaceutical composition includes an uncoupling protein 2 (UCP2) inhibitor and an interleukin-17 (IL-17) blockade.

Another embodiment of the present disclosure provides a method for promoting a cancer immunotherapy. The method includes administering an effective concentration of a pharmaceutical composition to a subject in need thereof, and the pharmaceutical composition includes an uncoupling protein 2 (UCP2) inhibitor and an interleukin-17 (IL-17) blockade.

### BRIEF DESCRIPTION OF THE DRAWINGS

The present disclosure can be more fully understood by reading the following detailed description of the embodiment, with reference made to the accompanying drawings as follows:

FIG. 1A, FIG. 1B, FIG. 1C, FIG. 1D, FIG. 1E, FIG. 1F, FIG. 1G, FIG. 1H, FIG. 1I, FIG. 1J and FIG. 1K show effects of UCP2 inhibition on cytotoxic T cell polarization.

FIG. 2A, FIG. 2B, FIG. 2C, FIG. 2D, FIG. 2E, FIG. 2F, FIG. 2G, FIG. 2H, FIG. 2I, FIG. 2J, FIG. 2K, FIG. 2L, FIG. 2M, FIG. 2N, FIG. 2O, FIG. 2P, FIG. 2Q, FIG. 2R and FIG. 2S show that UCP2 inhibition induces IFN-γ by enhancing T-bet through the up-regulation of OXPHOS and IL-12R/STAT4/mTOR axis in Tc1 cells.

FIG. 3A, FIG. 3B, FIG. 3C, FIG. 3D, FIG. 3E, FIG. 3F, FIG. 3G, FIG. 3H, FIG. 3I, FIG. 3J, FIG. 3K, FIG. 3L, FIG. 3M, FIG. 3N, FIG. 3O, FIG. 3P, FIG. 3Q, FIG. 3R, FIG. 3S, FIG. 3T and FIG. 3U show that UCP2 inhibition stimulates antitumor immunity but fails to suppress tumor progression in a pancreatic ductal adenocarcinoma (PDAC) model.

FIG. 4A, FIG. 4B, FIG. 4C, FIG. 4D, FIG. 4E, FIG. 4F, FIG. 4G, FIG. 4H, FIG. 4I, FIG. 4J, FIG. 4K, FIG. 4L and FIG. 4M show that depletion of IL-17 attenuates tumor growth and improves antitumor immunity in PDAC-bearing mice.

FIG. 5A, FIG. 5B, FIG. 5C, FIG. 5D, FIG. 5E, FIG. 5F, FIG. 5G, FIG. 5H, FIG. 5I, FIG. 5J, FIG. 5K, FIG. 5L, FIG. 5M and FIG. 5N show that the pharmaceutical composition of the present disclosure suppresses tumor growth in PDAC-bearing mice.

FIG. 6A, FIG. 6B, FIG. 6C, FIG. 6D, FIG. 6E, FIG. 6F, FIG. 6G, FIG. 6H, FIG. 6I, FIG. 6J, FIG. 6K, FIG. 6L, FIG. 6M and FIG. 6N show that the pharmaceutical composition of the present disclosure diminishes PDAC growth and enhances chemotherapy benefits against PDAC.

FIG. 7A, FIG. 7B, FIG. 7C, FIG. 7D, FIG. 7E and FIG. 7F show that the pharmaceutical composition of the present disclosure attenuates tumor growth and improves anti-tumor immunity in melanoma-bearing mice.

FIG. 8A, FIG. 8B, FIG. 8C, FIG. 8D, FIG. 8E, FIG. 8F, FIG. 8G and FIG. 8H show that UCP2 suppression facilitates IFN-γ production in peripheral blood and tumor infiltrating T cells retrieved from patients with PDAC.

FIG. 9 is a schematic view showing the pharmaceutical composition of the present disclosure enhancing antitumor immunity against PDAC.

### DETAILED DESCRIPTION

Unless defined otherwise, all scientific or technical terms used herein have the same meaning as those understood by persons of ordinary skill in the art to which the present disclosure belongs. Any method and material similar or equivalent to those described herein can be understood and used by those of ordinary skill in the art to practice the present disclosure.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in the specification and claims of the present disclosure are approximate and can vary depending upon the desired properties sought by the present disclosure.

The term “a/an” herein should mean one or more than one of the objects described in the present disclosure. The term “and/or” means either one or both of the alternatives.

The term “in vivo” herein generally means inside a living organism. The term “in vitro” generally means outside of a living organism, such as an experiment taking place at an artificial environment created outside of the living organism.

In the specification of the present disclosure, the gene name is represented by italic letters, the symbol format of its RNA is the same as that of the gene, and the protein related to the gene is represented by non-italic letters.

The terms “treatment,” “treating,” and “treat” herein generally refer to obtaining a desired pharmacological and/or physiological effect. The effect can be preventive in terms of completely or partially preventing a disease, a disorder, or a symptom thereof, and can be therapeutic in terms of a partial or complete cure for a disease, disorder, and/or symptoms attributed thereto. The term “treatment” used herein covers any treatment of a disease in a mammal (preferably a human) and includes suppressing development of the disease, the disorder, or the symptom thereof in a subject or relieving or ameliorating the disease, the disorder, or the symptom thereof in the subject.

The terms “individual,” “subject,” and “patient” herein are used interchangeably and refer to any mammalian subject for whom diagnosis, treatment, or therapy is desired.

The term “UCP2” refer to uncoupling protein 2, which is a mitochondrial membrane protein that regulates ATP synthesis. UCP2 is broadly expressed in lymph hematopoietic tissues, where increasing evidence suggests its immunomodulatory role. UCP2-deficiency promotes pathogen clearance by macrophages and worsens autoimmune disease progression in animal models of autoimmune diabetes and multiple sclerosis. Although the mechanisms remain uncertain, glycolysis-driven immunostimulation may play a primary role. In addition to the functional implication in immune cell activity, UCP2 is considered a strategy adopted by tumor cells to protect themselves from excessive reactive oxygen species (ROS).

The term “IL-17” refer to interleukin-17, which is a cytokine produced by T helper 17 (Th17) cells, promotes angiogenesis and facilitates both tumor growth and tumor invasion. Th17 cells accumulation has been observed in variety of malignancies and correlates to their poor prognosis.

A “cancer” in the specification refers to a physiological condition in a mammal characterized by a disorder of cell growth. A “tumor” includes one or more cancer cells. Examples of cancer include, but are not limited to, carcinoma, lymphoma, blastoma, sarcoma, and leukemia or lymphoid malignancies. More specific examples of such cancers include squamous cell carcinoma (e.g., epithelial squamous cell carcinoma), lung cancer (including small cell lung cancer, non-small cell lung cancer (NSCLC), lung adenoma, and lung squamous cell carcinoma), peritoneal cancer, hepatocellular carcinoma, gastric cancer (including gastrointestinal cancer), pancreatic cancer, pancreatic ductal adenocarcinoma (PDAC), melanomam, glioblastoma, cervical cancer, ovarian cancer, liver cancer, bladder cancer, hepatoma, breast cancer, colon cancer, rectal cancer, colorectal cancer, endometrial cancer or uterine cancer, salivary gland cancer, kidney cancer, prostate cancer, vulvar cancer, thyroid cancer, liver cancer, anal cancer, penile cancer, and head and neck cancer.

A “chemotherapeutic agent” in the specification is a chemical compound useful in the treatment of cancer, regardless of mechanism of action. Chemotherapeutic agents include compounds used in “targeted therapy” and conventional chemotherapy. Suitable chemotherapeutic agents can be selected from: agents that induce apoptosis; polynucleotides (e.g., ribozymes); polypeptides (e.g., enzymes); drugs; biological mimetics; alkaloids; alkylating agents; antitumor antibiotics; antimetabolites; hormones; platinum compounds; monoclonal antibodies conjugated with anticancer drugs, toxins, and/or radionuclides; biological response modifiers (e.g., interferons such as IFN-α and interleukins such as IL-2); adoptive immunotherapy agents; hematopoietic growth factors; agents that induce tumor cell differentiation (e.g., all-trans-retinoic acid); gene therapy reagents; antisense therapy reagents and nucleotides; tumor vaccines; and inhibitors of angiogenesis.

The present disclosure provides a pharmaceutical composition for promoting a cancer immunotherapy. The pharmaceutical composition includes an uncoupling protein 2 (UCP2) inhibitor and an interleukin-17 (IL-17) blockade.

The present disclosure also provides a method for promoting a cancer immunotherapy. The method includes administering an effective concentration of a pharmaceutical composition to a subject in need thereof, wherein the pharmaceutical composition includes an uncoupling protein 2 (UCP2) inhibitor and an interleukin-17 (IL-17) blockade.

In some embodiments, the UCP2 inhibitor can be a genipin, a Gardenia jasminoides Ellis or a siRNA targeting to an UCP2 gene. In some embodiments, the IL-17 blockade can be a monoclonal antibody targeting to IL-17, a recombinant anti-IL-17 receptor antibody (αIL-17RA) or a siRNA targeting to an IL-17A gene. In some embodiments, the monoclonal antibody targeting to IL-17 can be Secukinumab, ixekizumab or brodalumab. The pharmaceutical composition of the present disclosure can further include a chemotherapeutic agent. In some embodiments, the chemotherapeutic agent can be gemcitabine. In addition, the cancer immunotherapy can be for treating a pancreatic ductal adenocarcinoma or a melanoma.

The following specific examples are used to further illustrate the present disclosure, in order to benefit the person having ordinary skill in the art, and can fully utilize and practice the present disclosure without excessive interpretation. These examples should not be regarded as limiting the scope of the present disclosure, but is used to illustrate how to implement the materials and methods of the present disclosure.

### Materials and Methods

### 1. Mice

IL-17 knockout mice (kindly provided by Dr. Yoichiro Iwakura of University of Tokyo, Japan) were 7-10 weeks old when used for the experiments. C57 BL/6 mice were purchased from the National Laboratory Animal Center, Taipei, and housed in the Specific Pathogen Free room, Animal Center, China Medical University. Protocols were approved by the Animal Care and Use Committee of China Medical University, Taichung, Taiwan.

### 2. Cells

Human pancreatic cancer cell lines (MIA PaCa-2, Panc 1, and SU.86.86 cells) were cultured in the complete growth medium in a 37° C. humidified incubator supplied with 5% CO₂. PDAC cell line Pan18 (GFP-LUC-tagged) derived from pancreatic tumors in EKP (elastase-CreER; LSL-Kras^{G12D}; p53^{+/-}) C57BL/6 background mice was used for syngeneic mouse modeling. PDAC cell line 3036 (GFP-LUC-tagged) derived from pancreatic tumors in EKP (PDX-Cre; LSL-Kras^{G12D}; p53^{f/f}) C57BL/6 background mice was used for syngeneic mouse modeling.

### 3. In Vitro CD4 or CD8 T Cell Culture System

Mouse lymph nodes and spleens were harvested, and naïve CD4 or CD8 T cells isolated via magnetic bead separation (STEMCELL Technologies) and activated in α-CD3 (10 μg/ml) and α-CD28 (1 μg/ml) pre-coated 96-well plates. Skewing conditions were as follows: Th1 cells/Tc1 cells (3 ng/ml and 10 μ/ml α-IL-4), Th2 cells/Tc2 cells (20 ng/ml IL-4 and 10 μ/ml α-IFN-γ), Th17 cells/Tc17 cells (20 ng/ml IL-6, 1.25 ng/ml TGF-3, 20 ng/ml IL-1β, 20 ng/ml IL-23, 10 μg/ml α-IFN-γ and 10 μg/ml α-IL-4), and Treg cells (5 ng/ml TGF-β, 10 μg/ml α-IFN-γ and 10 μg/ml α-IL-4) in the indicated medium. Cells were cultured for 4-5 days followed by treatment with genipin (3.125 μM, 6.25 μM, 12.5 μM) or DMSO for 12-16 hours.

### 4. Surface Marker and Intracellular Cytokine Staining

Monoclonal antibodies were purchased from BD Biosciences or eBioscience. For intracellular cytokine staining (ICS), cells were first restimulated by PMA/ionomycin for 5 hours in the presence of GolgiStop (BD Biosciences), followed by surface epitope staining markers before being fixed/permeabilized (BD Cytofix/Cytoperm™) and stained with antibodies targeting intracellular antigens. Cells were finally fixed with 1.5% formaldehyde for 10 minutes at room temperature and analyzed on a FACSVerse flow cytometer (BD Bioscience). Data were analyzed using FlowJo software (Tree Star Inc.).

### 5. Gene Expression Analysis

For RNA-seq analysis, the differential gene expression analysis was conducted by RNA-seq quantification following the procedure of Illumina and was performed by Genomics (Taiwan). Low-quality bases and sequencing adapters in the raw data generated from the Illumina sequencer were removed, and the final library quality was assessed on the Agilent Bioanalyzer 2100 system. The gene expression profiles in the genipin-treated and control Tc1 cells were analyzed and compared. The differentially expressed genes were calculated by EBSeq, and functional analysis, such as (gene ontology) GO analysis, was performed using cluster Profiler. For real-time PCR, total RNA was extracted using the RNeasy Micro Kit (Qiagen), and cDNA was synthesized with the PrimeScript™ RT reagent kit (TakaRa). Real-time PCR amplification of IFN-γ was performed in triplicate using TaqMan Gene Expression Assay (Mm01168134_m1) in a Step One Plus system (Applied Biosystems). 18S primers were used as endogenous controls, and the relative gene expression was calculated using the 2^{ΔΔCt} method.

### 6. Immunoblotting

The protein concentrations of the total cell lysate or the extracted histones were determined by a Bradford assay (Bio-Rad Laboratories). Immunoblot analysis was performed with 10% sodium dodecyl sulfate-polyacrylamide gel electrophoresis, followed by overnight incubation with primary antibodies (1:1000 dilution) and incubation with horseradish peroxidase-conjugated secondary antibodies (1:5000 dilution; GeneTex). The polyvinylidene difluoride membranes were probed with antibodies against UCP2, pPI3K/PI3K, pAKT/AKT, pmTOR/mTOR, pSTAT1/STAT1, pSTAT4/STAT4, and H3K9Ac, which were purchased from Cell Signaling Technology or Genetex. Actin or Lamin B1 (Merck-Millipore) was used as the loading control. Signals were detected using an ECL solution (GE) and optical density quantified using ImageJ software.

### 7. Metabolic Assays

The real-time extracellular acidification rate (ECAR) and oxygen consumption rate (OCR) were measured using a Seahorse XF24 analyzer (Seahorse Bioscience), within XF Glycolysis Stress and XF Cell Mito Stress Test kits used to assess glycolysis and oxidative phosphorylation. 5×10⁵ cells exposed to the indicated treatments were plated in an XF-24 microplate coated with Cell-Tak (Corning) one day before the assay. ECAR and OCR were measured after metabolic manipulations by the addition of oligomycin, carbonyl cyanide-p-trifluoromethoxy-phenylhydrazone (FCCP), 2-deoxyglucose (2-DG) and rotenone/anti-mycin A (R/A). Calculations were conducted with Agilent Seahorse Wave Software for Agilent Seahorse XF analyzers (Seahorse Bioscience). For isolation of mitochondria, Tc1 cells were frozen and thawed to weaken the membranes and resuspended in Reagent A of a mitochondria isolation kit (ab110170, Abcam), before being homogenized and centrifuged at 1,000×g for 10 minutes at 4° C. Supernatant was saved (SN #1) and the pellet resuspended in Reagent B. After homogenization and centrifugation, supernatant was saved (SN #2). SN #1 and SN #2 were mixed thoroughly and centrifuged at 12,000×g for 15 minutes at 4° C. The pellet was collected in Reagent C supplemented with protease inhibitors and was used for the pyruvate and acetyl COA assays. Briefly, the mitochondrial pyruvate and acetyl CoA levels in the Tc1 cells were assessed using a commercial Pyruvate Assay Kit (ab65342, Abcam) and PicoProbe Acetyl COA Assay Kit (ab87546, Abcam) according to the manufacturer's instructions.

### 8. Histone H3 and H4 Total Acetylation

Histone extraction from the Tc1 cells was carried out using the EpiSeeker Histone Extraction Kit (ab113476; Abcam) according to the manufacturer's instructions. Samples were analyzed for histone H3 and histone H4 total acetylation using the H3/H4 Acetylation Detection Fast Fluorometric Kit (ab131561 and ab131562, Abcam), according to the manufacturer's instructions.

### 9. Soft Agar Colony Formation

Soft agar colony formation was analyzed by seeding 2,000-4,000 cells in a layer of 0.35% agar in complete growth medium over a layer of 0.5% agar in complete growth medium in the wells of a 12-well plate. 50 μl of serum-free media containing the genipin was replenished every 3 days. On days 14 or 21 after seeding, cells were fixed with crystal violet (Sigma-Aldrich C3886), and colonies counted under a light microscope.

### 10. Invasion Assay

Cells were seeded in the upper chamber with a Matrigel-coated membrane (Millicell Hanging Cell Culture Insert, PET 8 μm, 24-well, 48/pk; Millipore) in serum-free medium with or without genipin. Medium supplemented with serum was added to the lower chamber as a chemoattractant. After 48 hours or 72 hours of incubation, migrated cells were fixed with formaldehyde, stained with crystal violet and counted under a light microscope.

### 11. Tumoral Oxidative Stress and Apoptosis

The intracellular ROS, lipid peroxidation, nitric oxide (NO), and glutathione peroxidase (GPx) activities in the tumor cells were analyzed using the Reactive Oxygen Species (ROS) Detection Assay Kit (Biovision), lipid peroxidation was analyzed using the Lipid Peroxidation Colorimetric/Fluorometric Assay Kit (Biovision), Nitric Oxide Colorimetric Assay Kit (Biovision), and GPx assay kit (Biovision), respectively according to the manufacturer's instructions. The apoptosis rate was analyzed by FITC-Annexin V/PI kit (BD PharMingen) according to the manufacturer's instructions and performed on a flow cytometer using a FACSVerse instrument (BD Bioscience).

### 12. Tumorigenicity Assay in Mice

For Pan18-subcutaneous implantation, 5×10⁵ Pan18 cells were administered to wild-type (WT) or IL-17^{-/-} mice. Genipin (5 mg/kg, 10 mg/kg, or 30 mg/kg, i.p., every two days) was administered to Pan18-bearing mice for 3 weeks. For 3036-subcutanous implantation, 2×10⁵ 3036 cells were administered to WT mice. Genipin (10 mg/kg, i.p., every two days) and recombinant anti-IL-17 receptor antibody (αIL-17RA) (1 mg/kg, i.v., once a week) were administered to 3036-bearing mice for 3 weeks. Tumor volumes were evaluated every 2 days. For the orthotopic tumor modelling, mice pancreases were injected with 2×10⁵ Pan18 cells and monitored for tumor growth weekly using IVIS kinetics imaging system (Caliper Life-Sciences). Genipin (10 mg/kg, i.p., every two days) or αIL-17RA (1 mg/kg, i.v., once a week) was administered 1 week after tumor implantation. Mice were sacrificed 35 days after tumor induction and tumors weighed. For spontaneous pancreatic cancer model, Cre-dependent conditional knockin mutations of KrasG12D Kras⁺/LSLG12D mice (B6; 129-Kras2) were obtained from the Mouse Models of Human Cancer Consortium. Elas-CreERT; Kras⁺/LSLG12D mice was generated by breeding Kras⁺/LSLG12D mice with Elas-CreER mice obtained from the Level Transgenic Center. To induce the expression of Kras⁺/LSLG12D in acinar cells, 5-week-old Elas-CreERT; Kras⁺/LSLG12D male mice were injected with 100 μl of tamoxifen (20 mg/ml, i.p., three times per week) for 1 week. To induce chronic inflammation and tumors, 100 μl of cerulein (50 μg/ml, i.p., six times per week) (BACHEM) was given to the mice for 3 weeks. Then, the mice were injected with genipin (10 mg/kg, i.p., every two days) and αIL-17RA (1 mg/kg, i.v., once a week) until the end of the experiment, and overall survival was monitored. For genetically CD4/CD8-specific UCP2 deletion tumor modelling, we generated CD4Cre×UCP2^{f/f} mice by crossing loxP-UCP2-loxP mice with CD4Cre mice. CD4Cre×UCP2^{f/f}, WT, and loxP control mice underwent subcutaneous implantation of 2×105 Pan18 cells, followed by treatment with αIL-17RA (1 mg/kg, i.v., once a week) for 3 weeks. For melanoma modelling, IL-17/-and WT mice underwent subcutaneous implantation of 5×10⁵ B16F10 cells, followed by treatment with genipin (10 mg/kg, i.p., every two days) for 3 weeks. For CD8 depletion tumor modelling, IL-17^{-/-} mice underwent subcutaneous implantation of 2×10⁵ Pan18 cells, followed by treatment with genipin (10 mg/kg, i.p., every two days) and CD8 neutralizing antibody (5 mg/kg, i.v., once a week) for 3 weeks. Tumor volumes were evaluated every 3 days.

### 13. Serum Cytokines

Serum levels of IFN-γ, IL-17, TNF-α, IL-12, IL-6, and TGF-β were measured using commercial enzyme-linked immunosorbent assay (ELISA) kits (BD Biosciences; R&D systems; BioLegend) according to the manufacturer's protocol.

### 14. Chemokine Analysis

Total protein concentration of the tumor lysates was quantified by a Bradford assay. Tumoral chemokine profiles were determined by analyzing 250 μg protein from the tumor lysates using a Proteome Profiler™ Mouse Chemokine Array Kit (ARY020, R&D Systems) according to the manufacturer's instructions.

### 15. Immunohistochemistry

Immunohistochemical analysis was performed on formalin-fixed, paraffin-embedded tissues. Heat-induced antigen retrieval was performed using citrate buffer, pH 6.0, and the samples were autoclaved for 15 minutes. The slides were blocked with a CXCL10 (1:200, GTX31179; Genetex) antibody and a CCL2 (1:200, GTX81767; Genetex) antibody in PBS/5% FBS overnight at 4° C. and then developed [Ultra Vision Detection System Anti-Rabbit, HRP/DAB Kit (Thermo Fisher Scientific Inc.)]. Cellular nuclei of the sections were counterstained with hematoxylin, cleared and mounted using 3H-diethylphenylxanthine. Stained slides were analyzed by randomly capturing photomicrographs (Image-Pro Plus 4.0) at 6 different locations under a light microscope (Eclipse E600; Nikon Instech Co. Ltd.).

### 16. Study Subjects and Experimental Designs

PDAC human subject study was approved by the Research Ethics Committee of China Medical University & Hospital (CMUH109-REC2-148). 10 ml of fresh blood and 0.1-0.5 mm³ of tumor mass were collected on the same day from patients who underwent surgical resection. Monocytes were isolated from the patients' peripheral blood mononuclear cell (PBMC) or tumor mass. Cells were resuspended in RPMI medium containing 10% FBS and seeded at a density of 2×10⁵/well in a 12-well plate. Cells were treated with genipin (12.5 μM, 25 μM) and incubated overnight before staining for the flow cytometry analysis.

### 17. Statistical Analysis

All the data are presented as the means±SEMs, and Student's t test was used to compare the control and treatment groups. Group comparisons were performed using one-way analysis of variance and Tukey's honestly significant difference post hoc test. The marks of *, **, and *** on the statistical chart indicate statistical significance at p<0.05, p<0.01, and p<0.001, respectively.

### Results

### 1. UCP2 Inhibition Promotes Type I Effector Polarization

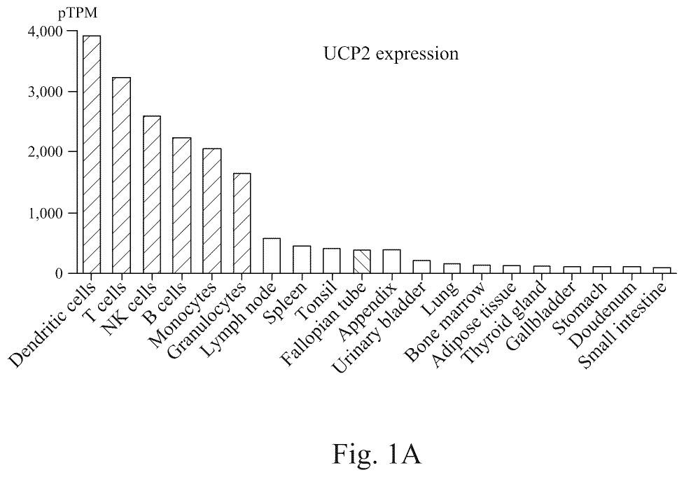

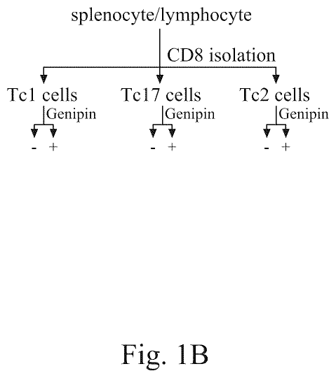

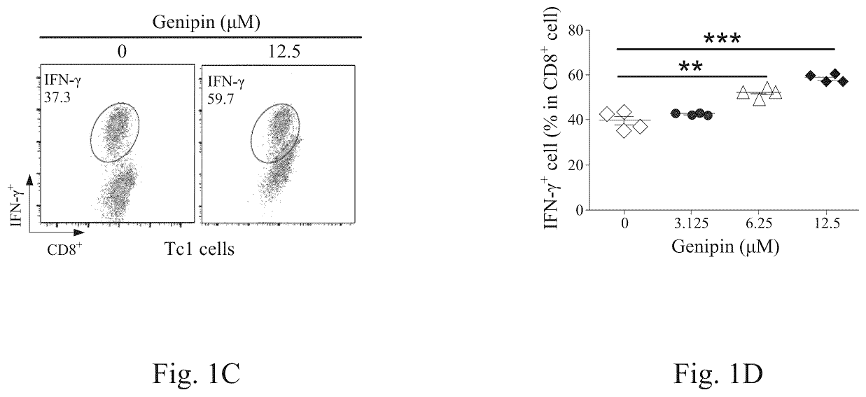

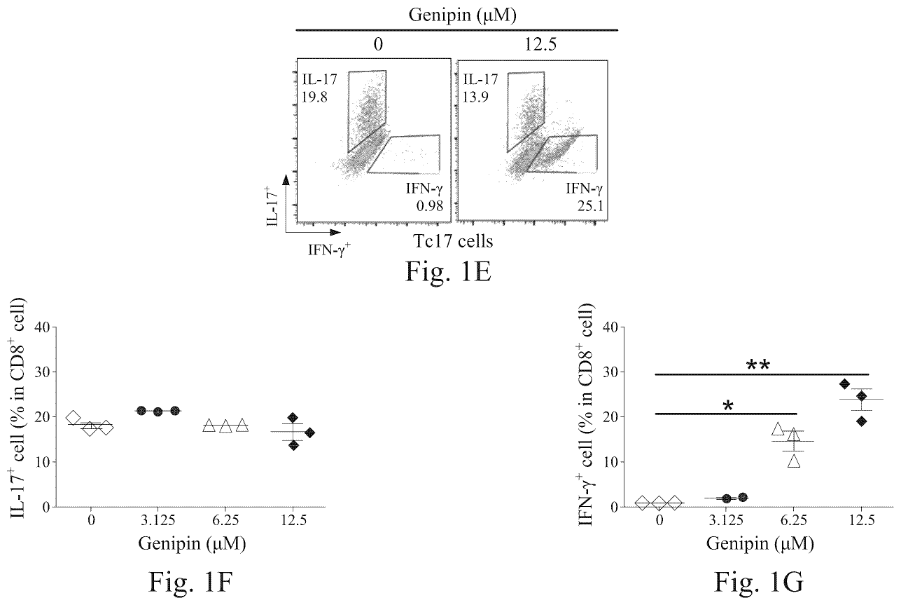

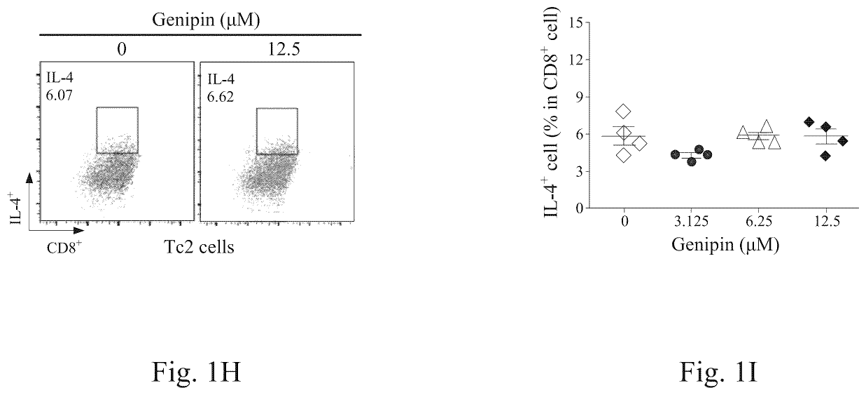

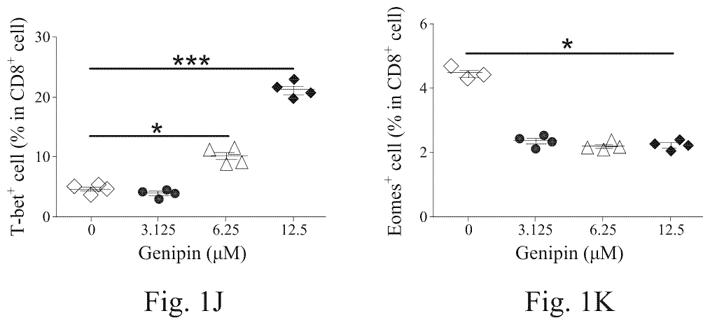

UCP2 inhibition was previously reported to enhance T cell responses, including antigen-driven T cell proliferation and production of TNF-α and IL-2 in CD4⁺ T cells during multiple sclerosis, and exacerbation of autoimmune diabetes. Reference is made to FIG. 1A, which shows UCP2 expression in different tissues or organs in human, and expression profiles for proteins in human tissues which were normalized to transcripts per million protein coding genes (pTPM) base on the Human Protein Atlas (HPA) database. Given broad UCP2 expression in immune cells, including T cells, the impact of UCP2 inhibition with genipin, an UCP2 inhibitor on cytotoxic and helper T cell polarization was evaluated. Reference is made to FIG. 1B to FIG. 1I, in which Tc1 represents Tc1 cells, Tc17 represents Tc17 cells, and Tc2 represents Tc2 cells. FIG. 1B shows an experimental schema of UCP2 inhibition in skewed Tc cells; FIG. 1C and FIG. 1D show representative flow cytometry dot plots and cumulative data showing IFN-γ production in genipin-treated Tc1 cells (N=4); FIG. 1E, FIG. 1F and FIG. 1G show representative flow cytometry dot plots and cumulative data showing IL-17 and IFN-γ production in genipin-treated Tc17 cells; and FIG. 1H and FIG. 1I show representative flow cytometry dot plots and cumulative data showing IL-4 production in genipin-treated Tc2 cells (N=4). The results in FIG. 1C to FIG. 1I show that UCP2 inhibition promoted IFN-γ in both Tc1 cells and Tc17 cells without impacting IL-17 expression in Tc17 cells, nor IL-4 expression in Tc2 cells.

Transcription factors Tbx21 (T-bet) and Eomes are the master regulators of type I effector differentiation, which drives IFN-γ transcription. Reference is made to FIG. 1J and FIG. 1K, which show intracellular cytokine staining (ICS) summary of T-bet (N=4) and Eomes (N=4) expression in the IFN-γ⁺CD8⁺ T cell population of genipin-treated Tc1 cells. The results in FIG. 1J and FIG. 1K show that UCP2 inhibition elevated the expression of T-bet but not Eomes in Tc1 cells. In addition, similarly to our observations in cytotoxic T cells can be observed in helper T cells (data not shown), UCP2 inhibition increased IFN-γ in both Th1 cells and Th17 cells without an impact on IL-17 expression in Th17 cells, nor significantly altering the populations of Th2 cells and regulatory T cells (Treg) among the groups. Thus, the suppression of UCP2 in T cells promotes anti-tumor immunity favorable type I effector and helper phenotypes in CD8 and CD4 T cells.

### 2. UCP2 Inhibition Augments IL-12R/STAT4/mTOR Axis and OXPHOS to Promote T-Bet-Driven IFN-γ Production in Tc1 Cells

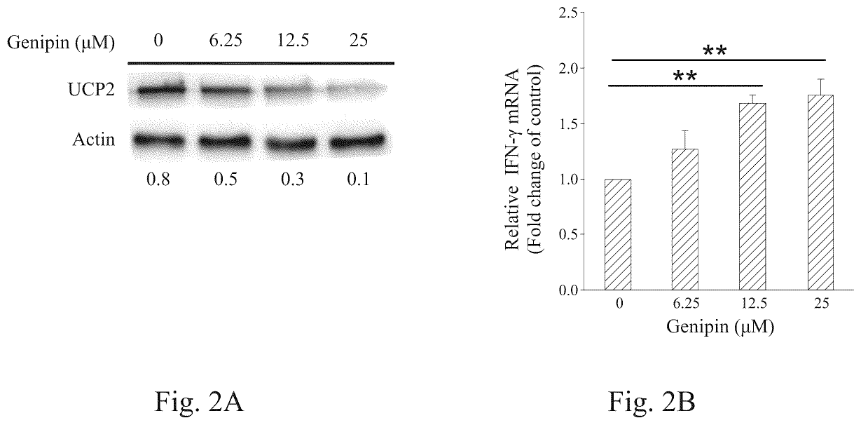

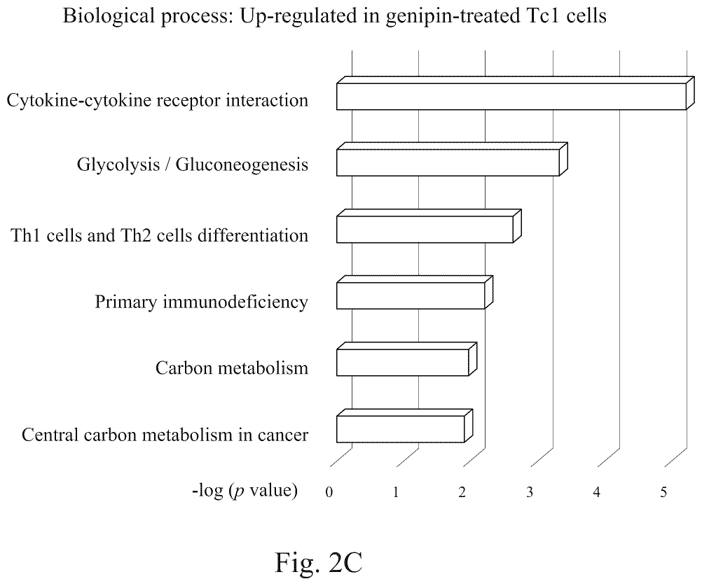

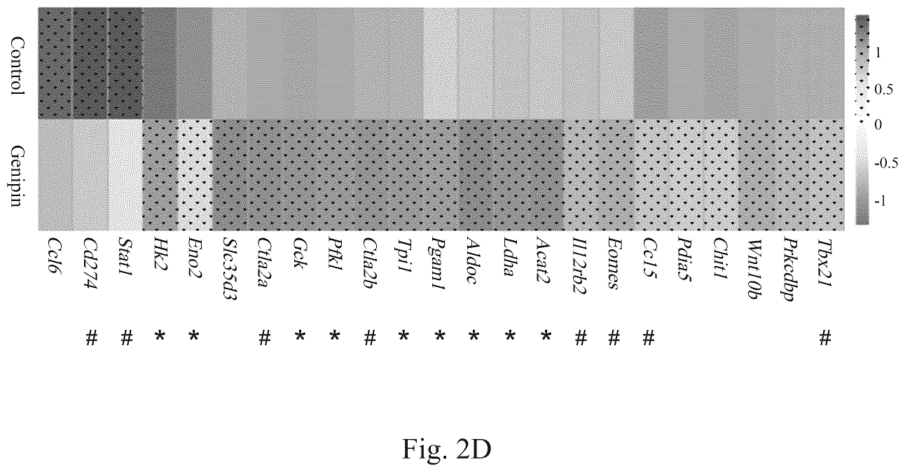

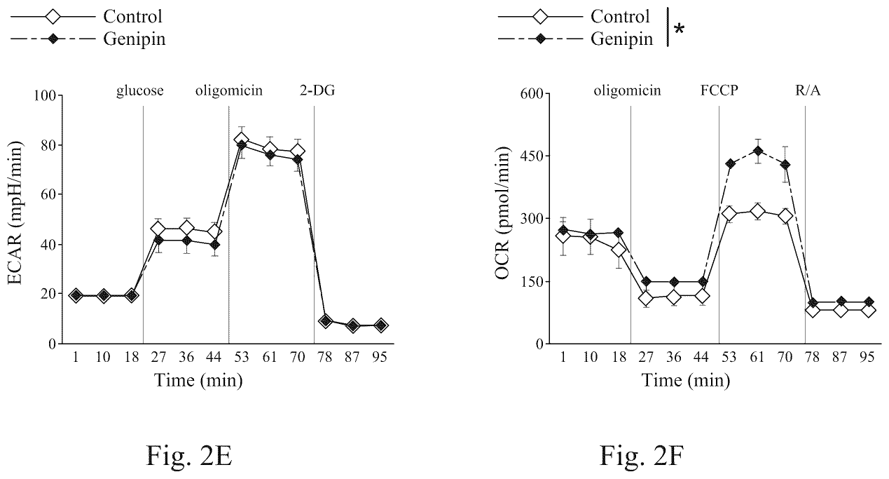

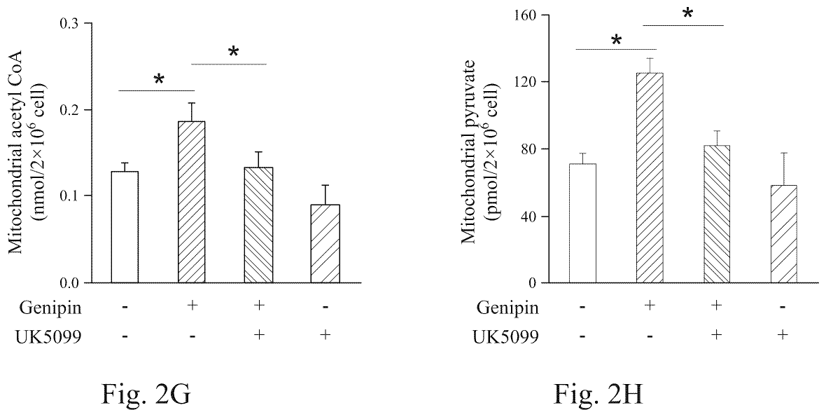

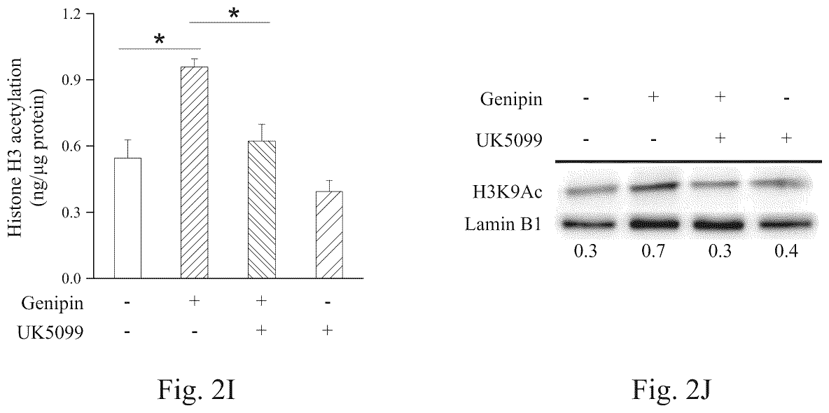

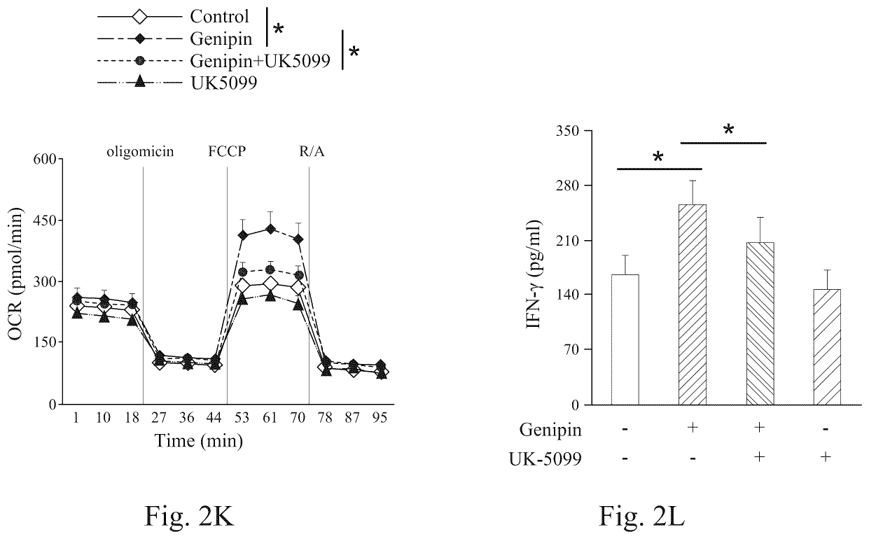

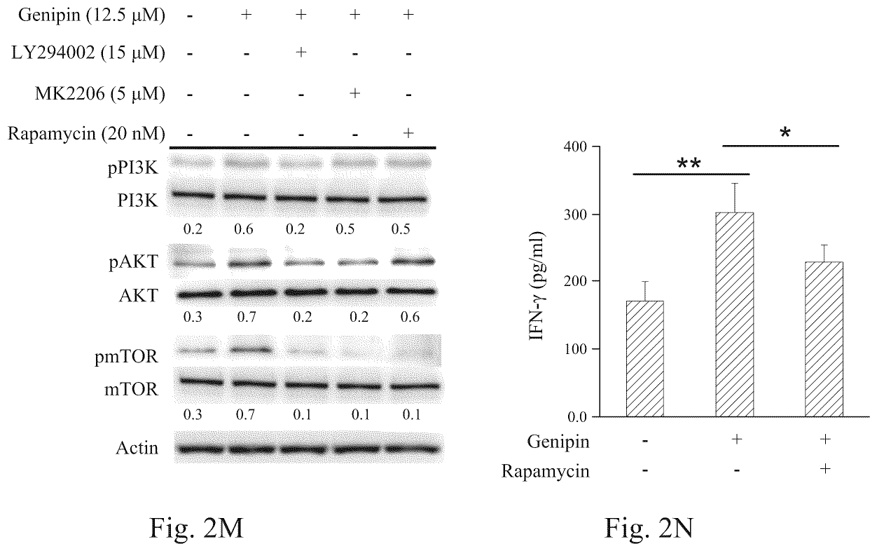

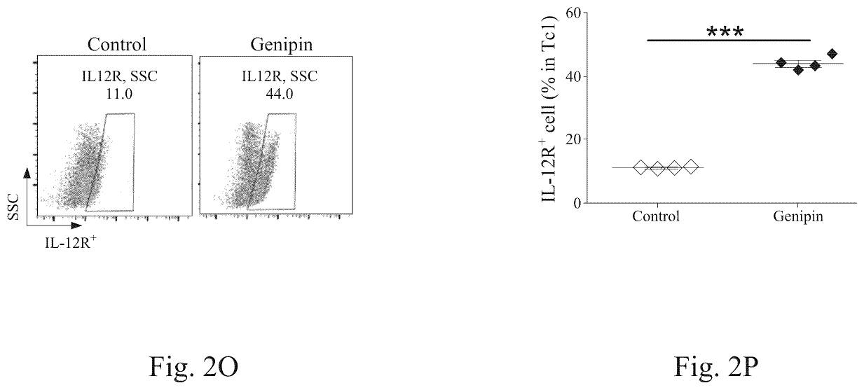

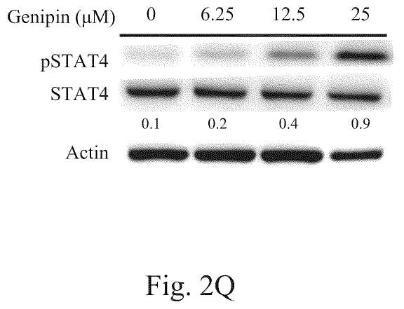

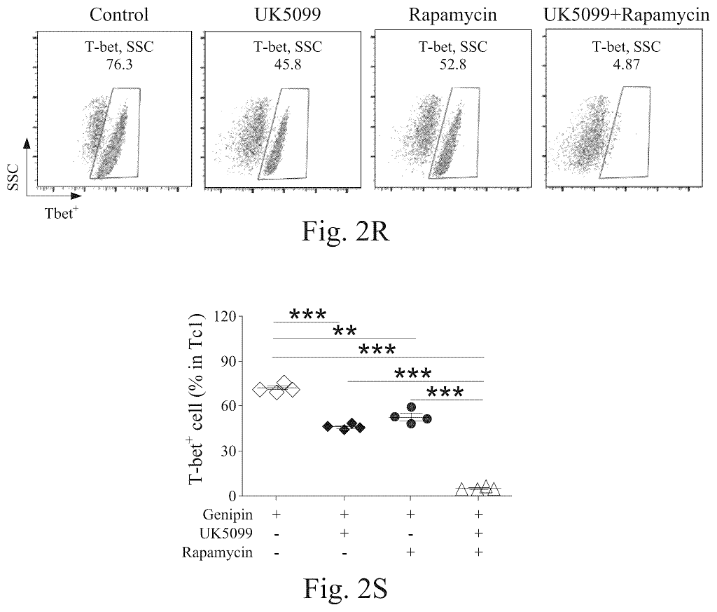

Next the transcriptional signature of Tc1 cells in response to the UCP2 inhibitor after confirming UCP2 expression and IFN-γ expression in the UCP2 inhibitor-treated Tc1 cells was assessed, and the UCP2 inhibitor used is genipin. Reference is made to FIG. 2A, which shows UCP2 expression in Tc1 cells treated with genipin for 12 hours, in which representative blotting of UCP2 is shown. Reference is made to FIG. 2B, which shows expression of IFN-γ mRNA in genipin-treated Tc1 cells (N=4). RNA-seq analysis identified 1495 differentially expressed genes in genipin-treated versus DMSO-treated Tc1 cells were analyzed. Reference is made to FIG. 2C, which shows that Gene Ontology Biological Process (GOBP) related to T cell activation and metabolism were enriched by UCP2 inhibition in Tc1 cells. P values of each GOBP were determined by clusterProfiler (R package) and converted to -logP to calculate the enrichment score in genipin (12.5 μM)-treated Tc1 cells. The results show that UCP2 inhibition correlated positively with expression of genes that control IFN-γ signaling, leukocyte activation and bioenergetic metabolism.

Reference is made to FIG. 2D, which shows list of genes significantly regulated by UCP2 inhibition in Tc1 cells related to T cell activation (#) and metabolism (*) selected by GOBP analysis. IFN-γ can be regulated on multiple levels to link cell metabolism to immune functions and an increased expression of genes critical in regulation of multiple levels of glycolytic pathway upon exposure to genipin can be found. These genes included Hk2, Gck, Pfkl, Aldoc, and Pgam1, as well as Eno2 and Ldha. These results indicate that UCP2 inhibition enables glycolysis and facilitated pyruvate production in Tc1 cells. In addition, genipin-treated Tc1 cells augmented the expression of genes related to T cell activation and migration, including Tbx21, Ccl5, Eomes, Il12rb2, Ctla2a and Ctla2b. Reference is made to FIG. 2E and FIG. 2F, which show bioenergetics analysis of Tc1 cells treated with genipin (12.5 μM), and the results of ECAR (N=5) and OCR (N=5) were measured by Seahorse XF24 analyzer. Surprisingly, despite upregulation of genes encoding glycolytic machinery, UCP2 inhibition had no functional impact on metabolic glycolysis readouts (normal ECAR), whereas it promoted oxidative phosphorylation (OCR) in Tc1 cells. To address this, the main source of TCA cycle-acetyl COA in the whole cell and the mitochondria was examined. Reference is made to FIG. 2G to FIG. 2L, which shows effect of mitochondrial pyruvate transport on metabolism in genipin-treated Tc1 cells, and the results of mitochondrial acetyl COA pyruvate (N=4), mitochondrial pyruvate (N=4), histone H3 acetylation (N=4), H3K9 acetylation, OCR (N=5), and IFN-γ (N=4) were evaluated in genipin-treated Tc1 cells with or without UK5099 (40 μM). UK5099 is a specific mitochondrial pyruvate carrier (MPC) inhibitor. The results show that genipin increased the level of acetyl CoA in both the mitochondria (FIG. 2G) and the cytosol (data not shown), with parallel increase in mitochondrial pyruvate (FIG. 2H), whereas cytosolic fraction was decreased in genipin-treated Tc1 cells (data not shown). Mitochondrial pyruvate uptake links cytosolic glycolysis and mitochondrial oxidative phosphorylation (OXPHOS), with the process regulated by the MPC, a complex formed by the MPC1 and MPC2 subunits located on the inner membrane of mitochondria. Genipin-driven Tc1 effects could be reversed was further demonstrated using UK5099 (FIG. 2G to FIG. 2L), and histone H3 acetylation (H3K9Ac) was observed (FIG. 2I and FIG. 2J). Taken together, these results suggest that upon UCP2 inhibition, mitochondrial TCA can enhance acetyl CoA generation to promote histone acetylation of the IFN-γ loci, leading to the increased IFN-γ production in Tc1 cells.

As T-bet-dependent heritable type I effector proliferation and maturation is dependent upon the IL-12/IL-12 receptor (IL-12R) signaling mediating to sustained mammalian target of rapamycin (mTOR) activity via the phosphoinositide 3-kinase (PI3K) and signal transducer and activator of transcription 4 (STAT4) pathways, the effects of UCP2 inhibition on these signaling effects was next evaluated. The expression of mTOR and its upstream kinases PI3K and AKT was increased in genipin-treated To1 cells. To further query the role of the mTOR signaling axis in the Tc1 cells T-bet activation, different pathway inhibitors were used, including LY294002, MK2206, and rapamycin, in genipin-treated Tc1 cells. Reference is made to FIG. 2M to FIG. 2S. FIG. 2M shows effect of mTOR inhibitors on PI3K, AKT, and mTOR levels in genipin-treated Tc1 cells, in which representative blots are shown. FIG. 2N, which shows effect of rapamycin on IFN-γ levels in genipin-treated Tc1 cells. FIG. 2O and FIG. 2P show representative flow cytometry dot plots and cumulative data showing IL-12R expression in genipin (12.5 μM)-treated Tc1 cells, in which FIG. 2P shows the statistical results of FIG. 2O. FIG. 2Q shows STAT4 levels in genipin-treated Tc1 cells, in which Tc1 cells were incubated with genipin for 12 hours, and representative blotting is shown. FIG. 2R and FIG. 2S show effect of UK5099 and rapamycin on T-bet expression in genipin-treated Tc1 cells (N=4), in which representative flow cytometry dot plots and cumulative data are shown, and FIG. 2S shows the statistical results of FIG. 2R. The results in FIG. 2M shows that the mTOR blockade specifically decreased the genipin-induced PI3K expression, AKT expression, and mTOR expression. Notably, IFN-γ expression was significantly suppressed by rapamycin in genipin-treated Tc1 cells (FIG. 2N). In addition to the upregulated level of the Il12rb2 gene observed in the RNA-seq analysis, UCP2 inhibition also increased IL-12R expression in Tc1 cells (FIG. 2O and FIG. 2P), a finding also associated with STAT4 activation (FIG. 2Q). Furthermore, whether mTOR regulation of T-bet was responsible for the observed rapamycin-induced IFN-γ suppression in genipin-treated Tc1 cells was tested. Interestingly, both mTOR inhibitions with rapamycin and MPC1 blockade with UK5099 blocked the positive regulation of T-bet in UCP2-deprived Tc1 cells, with a synergistic effect of a combined rapamycin and UK5099 treatment (FIG. 2R and FIG. 2S). In summary, UCP2 inhibition enhances metabolic functioning of Tc1 cells in an IL-12/STAT4/mTOR-dependent fashion to augment their immune fitness provide immunostimulatory.

### 3. UCP2 Inhibition Alone is Insufficient to Suppress Tumor Growth in PDAC Models

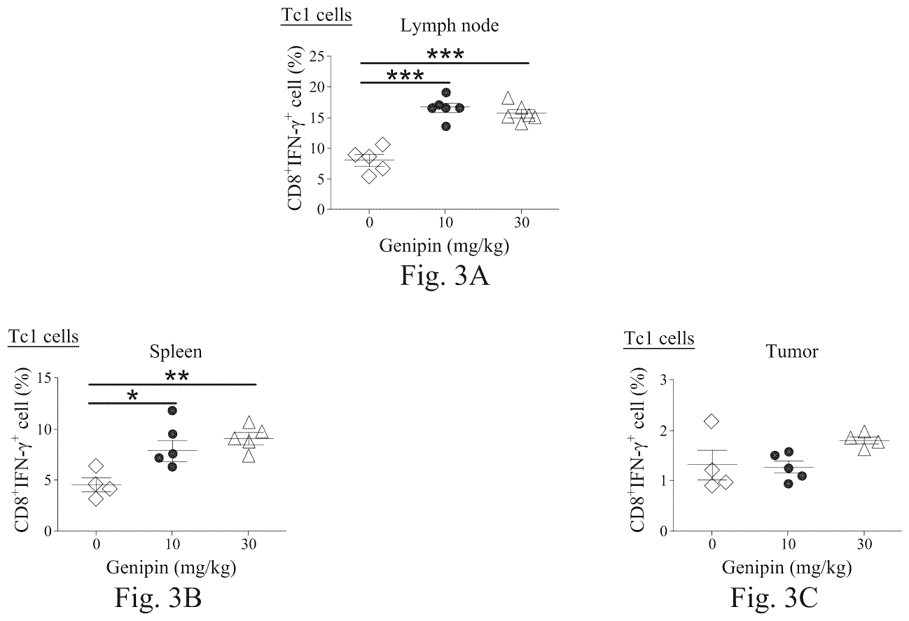

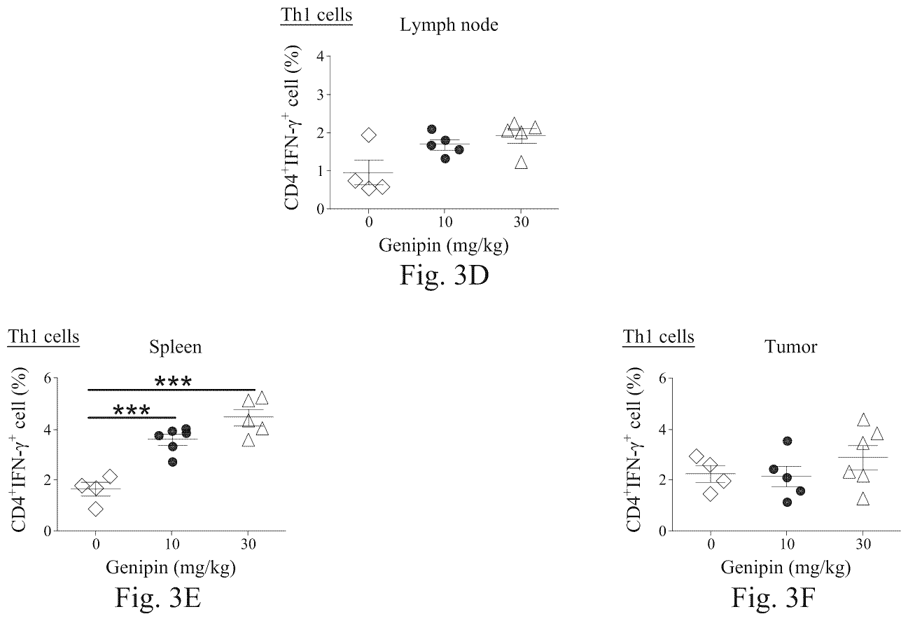

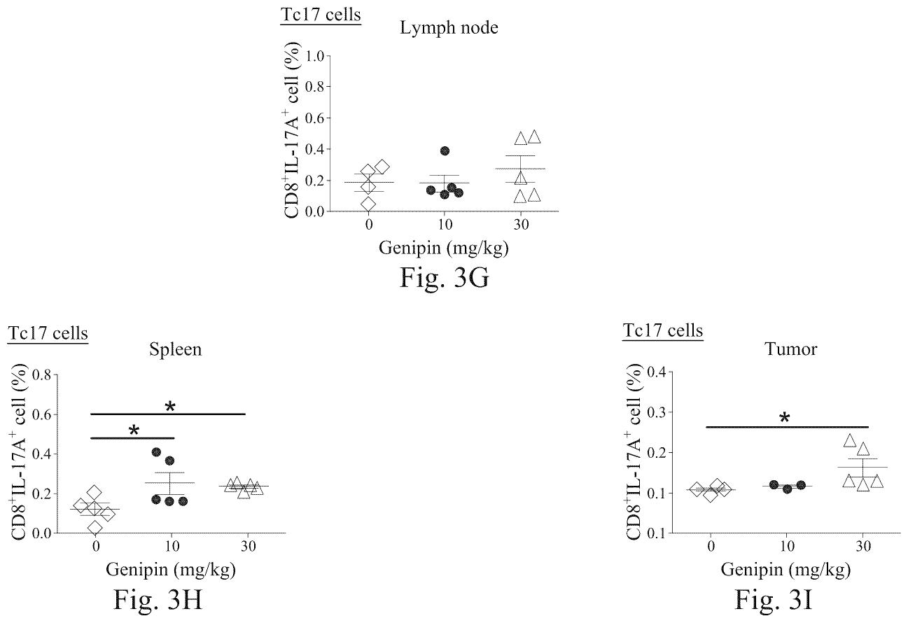

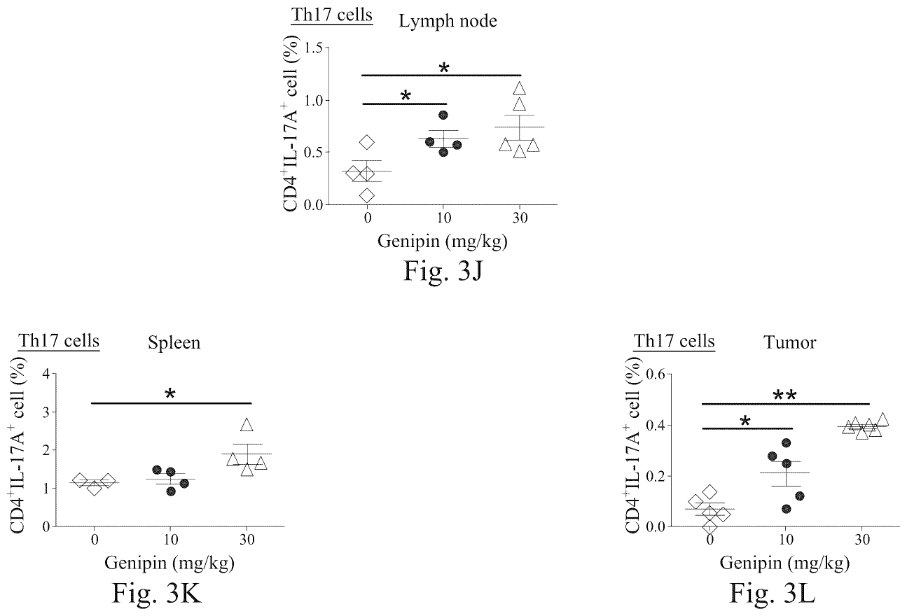

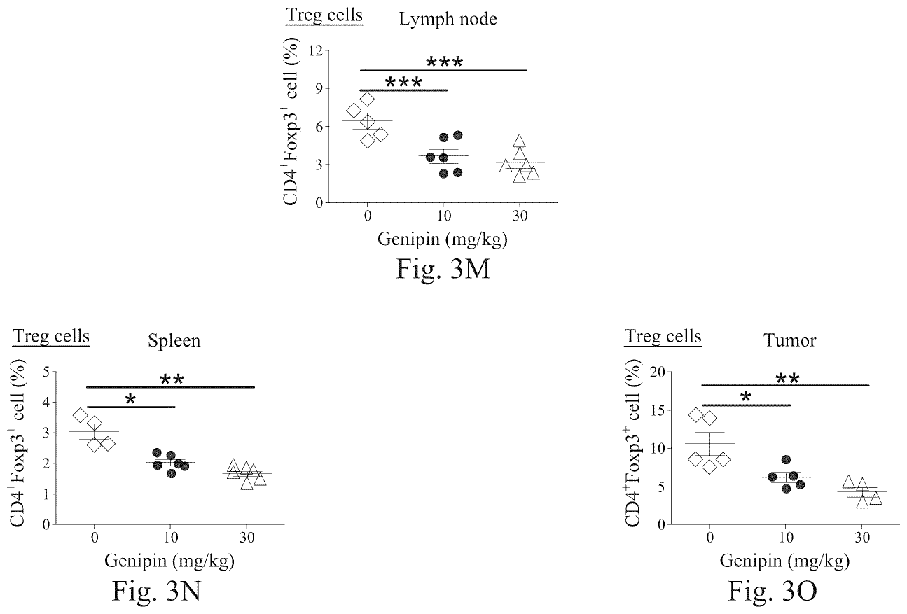

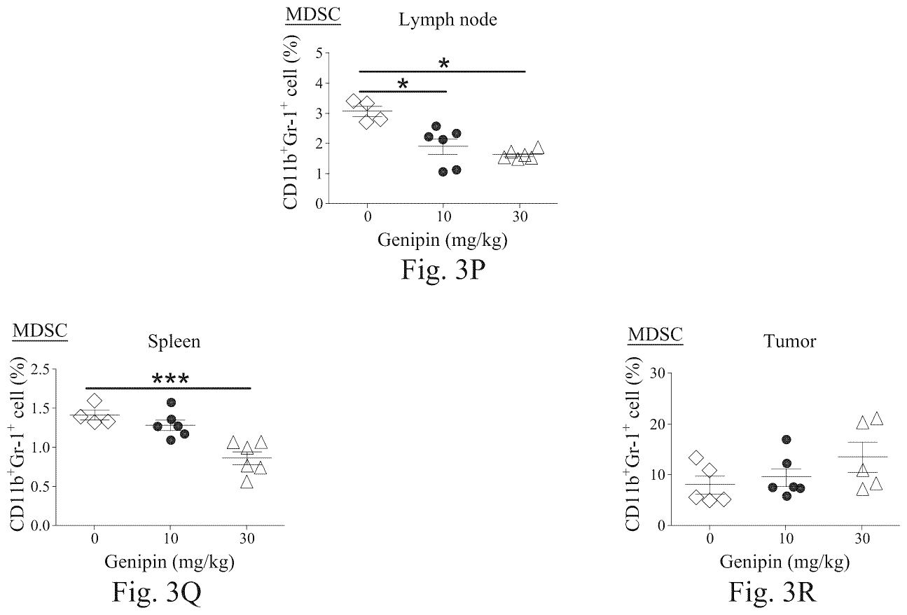

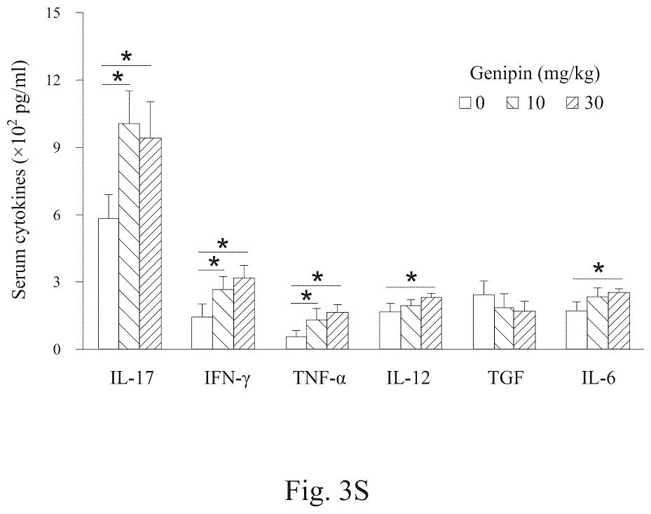

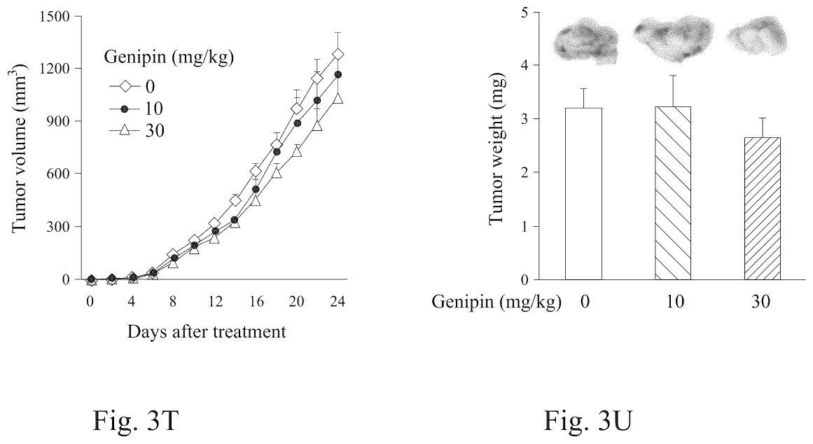

Given in vitro immunostimulatory effects with genipin use, in vivo impact of UCP2 inhibition in an ectopic model of mouse PDAC was next examined. To analyze tumor immunity, different types of immune cells were further characterized, including Tc1 cells, Th1 cells, Tc17 cells, Th17 cells, myeloid-derived suppressor cells (MDSC), and Treg cells in spleen, tumor-draining lymph nodes (hereinafter referred to as lymph node), and tumor tissues (hereinafter referred to as tumor) of PDAC-bearing mice. Reference is made to FIG. 3A to FIG. 3R, which show analysis results of flow cytometry. FIG. 3A, FIG. 3B and FIG. 3C show populations of Tc1 cells in lymph node, spleen and tumor, respectively. FIG. 3D, FIG. 3E and FIG. 3F show populations of Th1 cells in lymph node, spleen and tumor, respectively. FIG. 3G, FIG. 3H and FIG. 3I show populations of Tc17 cells in lymph node, spleen and tumor, respectively. FIG. 3J, FIG. 3K and FIG. 3L show populations of Th17 cells in lymph node, spleen and tumor, respectively. FIG. 3M, FIG. 3N and FIG. 3O show populations of Treg cells in lymph node, spleen and tumor, respectively. FIG. 3P, FIG. 3Q and FIG. 3R show populations of MDSC in the lymph node, spleen and tumor, respectively. In addition to the increases in the absolute numbers of Tc1 cells and Th1 cells, the numbers of Tc17 cells and Th17 cells were increased in genipin-treated tumor-bearing mice (FIG. 3A to FIG. 3L). In contrast, genipin decreased the numbers of Treg cells and MDSC in the lymph nodes and spleens of PDAC-bearing mice, suggesting that UCP2 inhibition not only has direct immunostimulatory effects on effector T cells but can also tilt the balance in favor of anti-cancer responses through the elimination of immunosuppressive milieu (FIG. 3M to FIG. 3R). Notably, no changes in the tumor infiltrating Tc1 cells, Th1 cells, and MDSC in genipin-treated PDAC-bearing mice were observed (FIG. 3A to FIG. 3F and FIG. 3P to FIG. 3R).

Furthermore, reference is made to FIG. 3S to FIG. 3U. FIG. 3S shows serum IL-17, IFN-γ, TNF-α, IL-12, TGF, and IL-6 levels measured at the end of the experiment. FIG. 3T and FIG. 3U show tumorigenesis assay in genipin-treated PDAC-bearing mice, in which mice were implanted subcutaneously with Pan18 cells (N=6). Tumor volume in FIG. 3T was measured every two days, and tumor weight in FIG. 3U assessed at the end of the experiment. Using escalating doses of genipin, UCP2 treatment increased serum IL-17, IFN-γ, TNF-α, IL-12, and IL-6 in PDAC-bearing mice (FIG. 3S). However, despite the encouraging changes in the cellular immune landscape, UCP2 inhibition did not suppress tumorigenesis in PDAC-bearing mice (FIG. 3T and FIG. 3U). Taken together, these data suggest that UCP2 inhibition alone, despite ability to enhance key effector T cell responses, is an insufficient immunotherapeutic modality in an established model of PDAC.

### 4. The Pharmaceutical Composition of the Present Disclosure Suppress Tumor Growth in Tumor-Bearing Mice

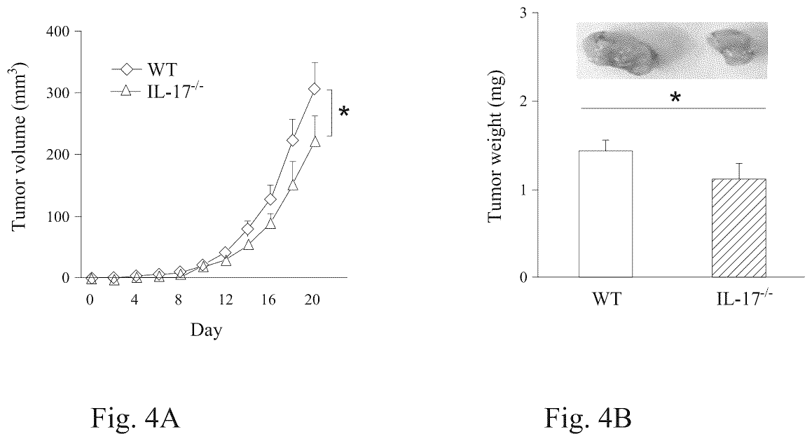

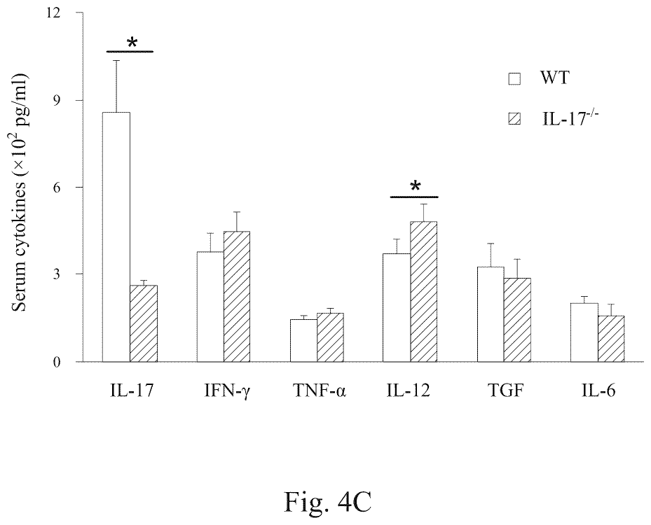

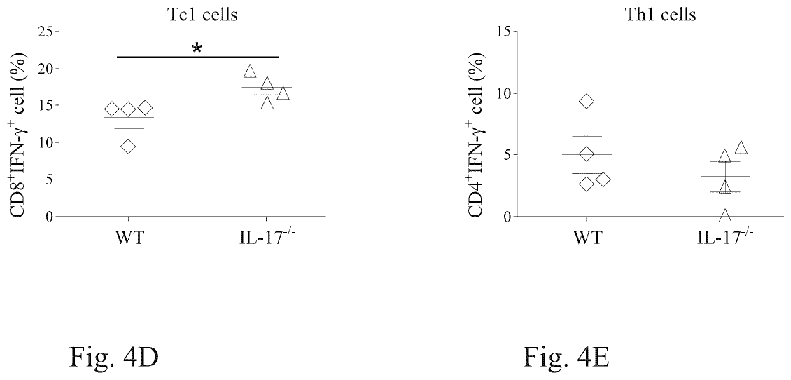

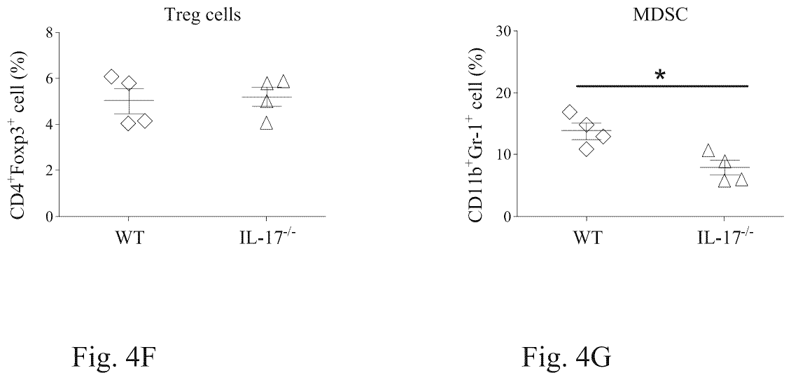

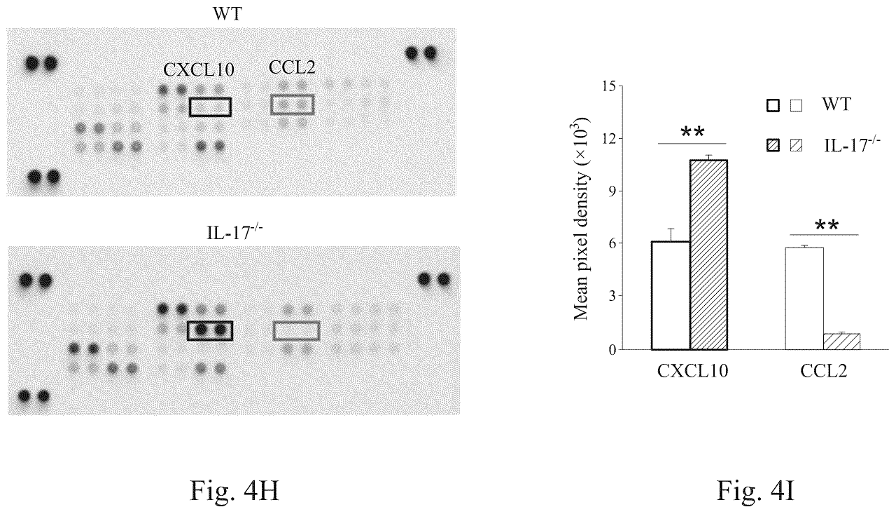

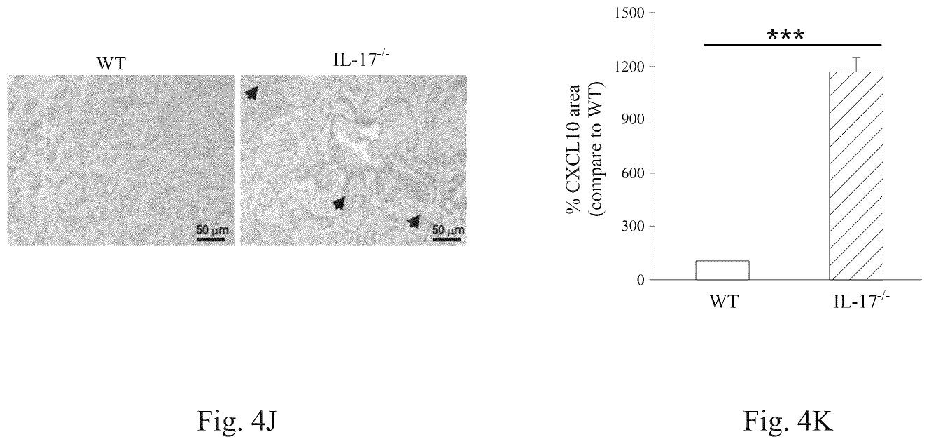

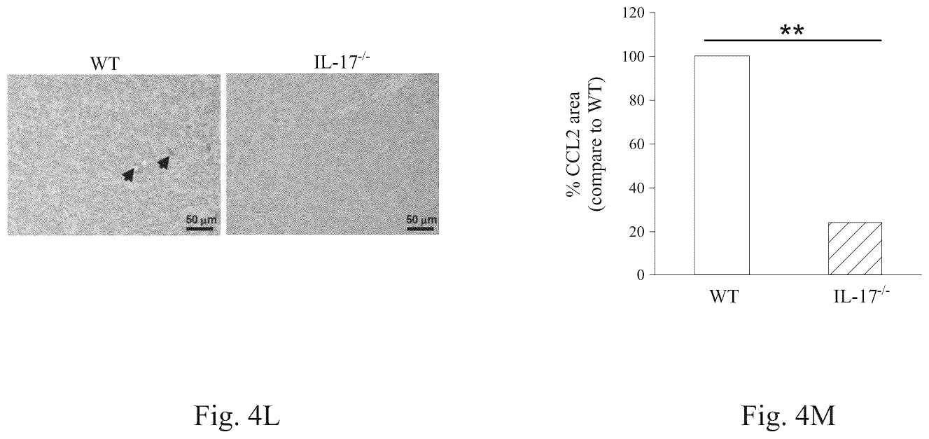

In this part, the effect of the pharmaceutical composition of the present disclosure on suppressing tumor growth in tumor-bearing mice is further demonstrated. Changes in tumor immunogenicity and inhibition of immune response are crucial mechanisms that allow tumors to progress to an advanced stage. IL-17 overexpressing pancreatic cancers exhibit a more aggressive course, whereas suppression of IL-17 enhances PDAC-directed antitumor immunity in a process associated with enhanced IFN-γ secretion. Reference is made to FIG. 4A and FIG. 4B, which show tumorigenesis assay in PDAC-bearing wild-type mice (represented as WT) or PDAC-bearing IL-17^{-/-} mice (represented as IL-17^{-/-}). Mice were implanted subcutaneously with Pan18 cells (N=4). Genipin (5 mg/kg or 10 mg/kg, i.p., every two days) was administered to PDAC-bearing IL-17^{-/-} mice for 3 weeks. Tumor volume in FIG. 4A was measured every two days, and tumor weight in FIG. 4B assessed at the end of the experiment. In FIG. 4A and FIG. 4B, IL-17 depletion alone modestly inhibited tumor growth was found. Based upon these observations, combined UCP2 inhibition and IL-17 blockade can exhibit therapeutic synergy and enhance antitumor immunity in preclinical PDAC models was hypothesized. Reference is made to FIG. 4C to FIG. 4G, which show results of systemic and local tumor immunity in PDAC-bearing mice, including PDAC-bearing WT mice (represented as WT) and PDAC-bearing IL-17^{-/-} mice (represented as IL-17^{-/-}). Serum IL-17, IFN-γ, TNF-α, IL-12, TGF, and IL-6 levels in FIG. 4C were measured at the end of the experiment. Populations of Tc1 cells in PDAC-bearing mice in FIG. 4D, populations of Th1 cells in PDAC-bearing mice in FIG. 4E, populations of Treg cells in PDAC-bearing mice in FIG. 4F, and populations of MDSC in PDAC-bearing mice in FIG. 4G were assessed by flow cytometry. In PDAC-bearing IL-17^{-/-} mice, increased serum IL-12 and increased numbers of splenic and tumoral Tc1 cells were observed, whereas the numbers of splenic and tumoral MDSC were decreased (FIG. 4C to FIG. 4G).

The chemokine-driven recruitment of polyfunctional immune cells into the tumor microenvironment determines the protumor or antitumor nature of the environment. Therefore, different chemokines in the tumor tissue of PDAC-bearing IL-17^{-/-} mice or PDAC-bearing WT mice were screened by chemokine array. Reference is made to FIG. 4H to FIG. 4M, which show tumor chemokine levels in PDAC-bearing mice, including PDAC-bearing WT mice (represented as WT) and PDAC-bearing IL-17^{-/-} mice (represented as IL-17^{-/-}). Chemokine array in FIG. 4H and FIG. 4I was performed in tumor lysates of PDAC-bearing mice, in which FIG. 4I shows the statistical results of FIG. 4H. Representative immunohistochemistry (IHC) analysis and quantitative results of CXCL10 were shown in FIG. 4J and FIG. 4K and that of CCL2 were shown in FIG. 4L and FIG. 4M in PDAC-bearing mice. FIG. 4K shows the statistical results of FIG. 4J, and FIG. 4M shows the statistical results of FIG. 4L. Notably, both chemokine array and IHC analysis revealed that IL-17 depletion significantly elevated the CXCL10 levels and suppressed the CCL2 levels in PDAC-bearing mice (FIG. 4H to FIG. 4M), providing a possible mechanistic link to previous observations.

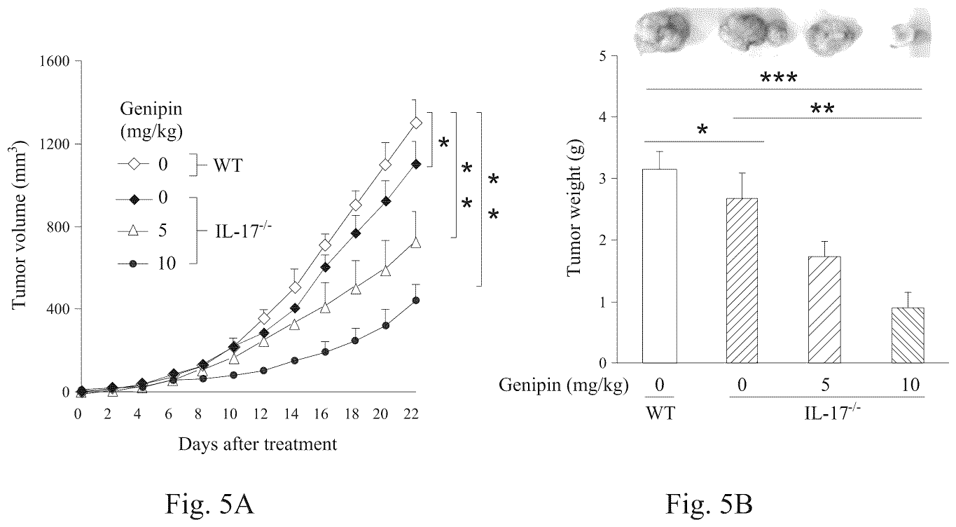

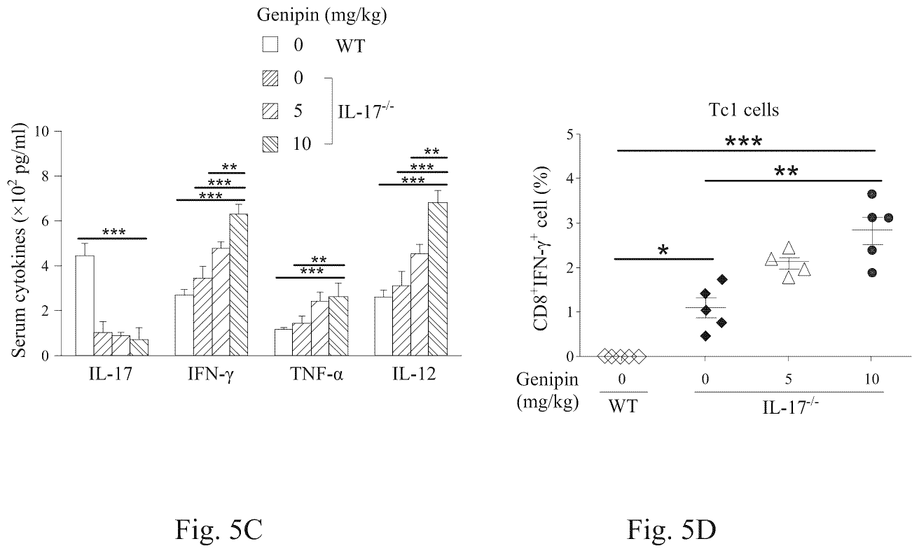

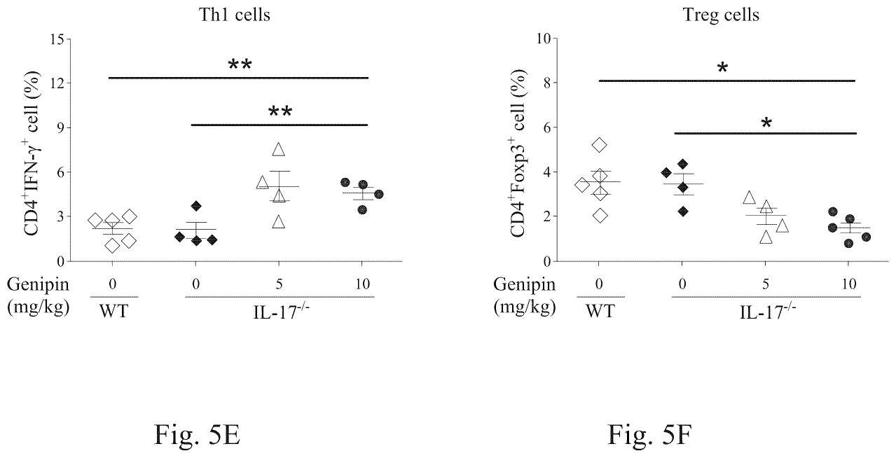

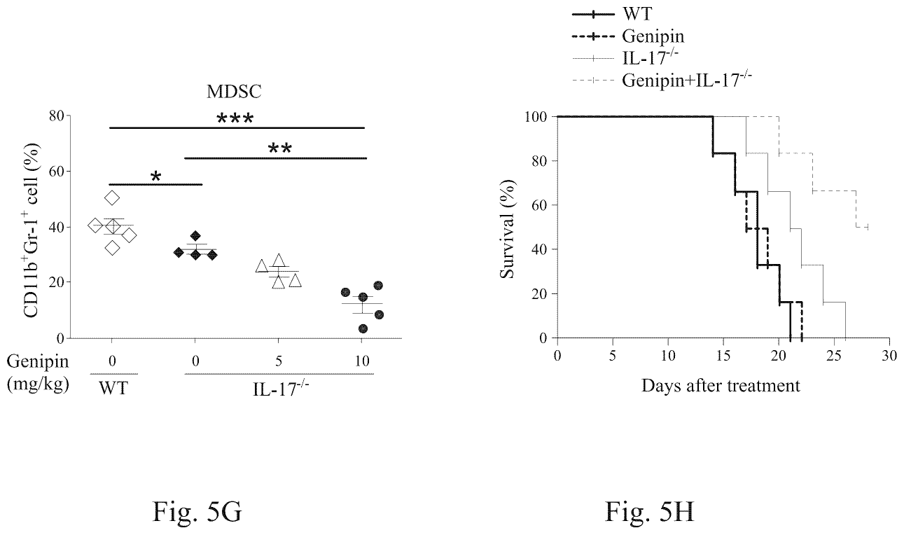

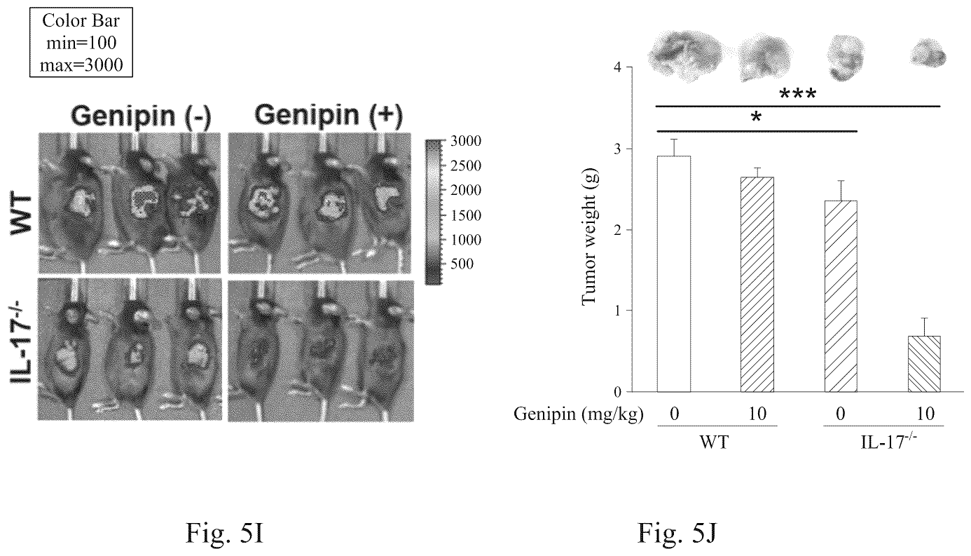

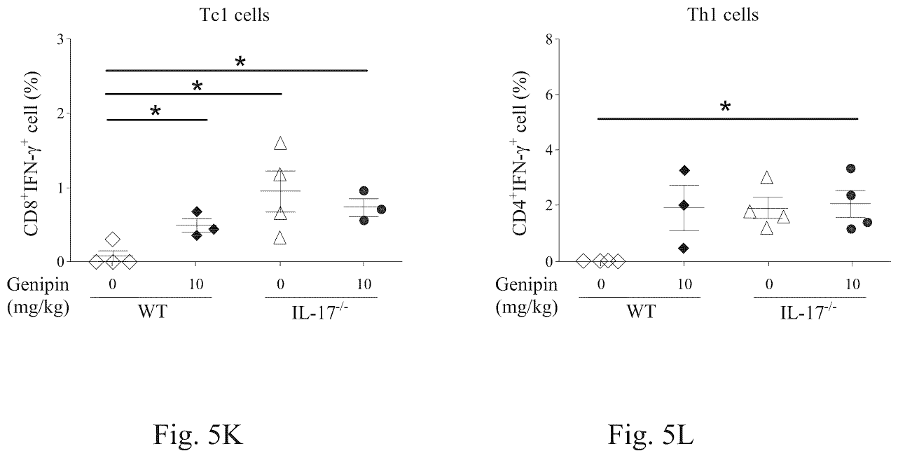

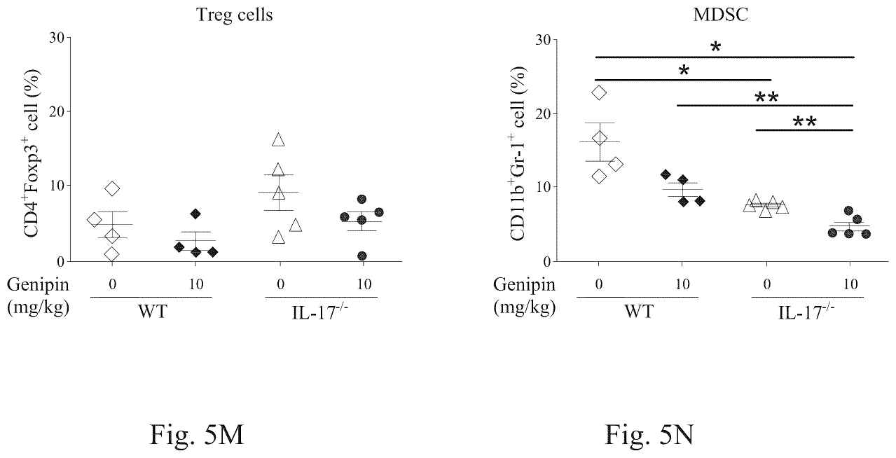

Next, combinatorial approaches to target UCP2 and IL-17 simultaneously were focused on and subcutaneous and orthotopic PDAC-modeling strategies were examined. Reference is made to FIG. 5A and FIG. 5B, which show the results of tumorigenesis assay in genipin-treated PDAC-bearing IL-17^{-/-} mice (N=4-5). Mice were implanted with Pan18 cells. Genipin (5 mg/kg or 10 mg/kg, i.p., every two days) was administered to PDAC-bearing IL-17^{-/-} mice for 3 weeks. Tumor volume in FIG. 5A was measured every two days, and tumor weight in FIG. 5B assessed at the end of the experiment. In FIG. 5A and FIG. 5B, the combined treatment provided a synergistic effect to inhibit tumor growth in PDAC-bearing mice.

The serum cytokines and the populations of Tc1 cells, Th1 cells, MDSC, and Treg cells in PDAC-bearing mice are further assessed. Reference is made to FIG. 5C to FIG. 5G, which show the results of systemic and local tumor immunity in genipin-treated PDAC-bearing mice (N=4-5), including PDAC-bearing WT mice (represented as WT) and PDAC-bearing IL-17^{-/-} mice (represented as IL-17^{-/-}. Serum IL-17, IFN-γ, TNF-α, and IL-12 levels in FIG. 5C were measured. Populations of Tc1 cells in PDAC-bearing mice in FIG. 5D, populations of Th1 cells in PDAC-bearing mice in FIG. 5E, populations of Treg cells in PDAC-bearing mice in FIG. 5F, and populations of MDSC in PDAC-bearing mice in FIG. 5G were assessed by flow cytometry. Concordant with previous observations with UCP2 inhibition in PDAC-bearing WT mice, genipin increased the serum IFN-γ, IL-12, and TNF-α levels in PDAC-bearing IL-17^{-/-} mice in a dose-dependent manner. An additional effect was reflected in the increased numbers of Tc1 cells and the decreased numbers of MDSC in genipin-treated PDAC-bearing IL-17/mice (FIG. 5D to FIG. 5G), whereas UCP2 inhibition had no effects on IL-17-ablation driven changes in CXCL10 and CCL2 levels in PDAC-bearing mice (data not shown).

Orthotopic PDAC model was used to validate benefits of immunotherapeutic UCP2 inhibition combined with IL-17 blockade. Reference is made to FIG. 5H to FIG. 5J, which show the results of survival, bioluminescence monitoring, and tumor weight of genipin-treated PDAC-bearing WT mice or PDAC-bearing IL-17^{-/-} mice following orthotopic Pan18 implantation. PDAC-bearing WT mice or PDAC-bearing IL-17^{-/-} mice underwent orthotopic implantation of Pan18 cells (N=5). Genipin (10 mg/kg, i.p., every two days) was administered for 3 weeks. The results of survival (N=6) in FIG. 5H, bioluminescent tumor growth monitoring (N=5) in FIG. 5I, and tumor weights (N=5) in FIG. 5J were compared. Reference is made to FIG. 5K to FIG. 5N, which show the results of tumor immunity in genipin-treated PDAC-bearing mice, including PDAC-bearing WT mice (represented as WT) or PDAC-bearing IL-17^{-/-} mice (represented as IL-17^{-/-}). Populations of Tc1 cells in PDAC-bearing mice in FIG. 5K, populations of Th1 cells in PDAC-bearing mice in FIG. 5L, populations of Treg cells in PDAC-bearing mice in FIG. 5M, and populations of MDSC in PDAC-bearing mice in FIG. 5N were assessed by flow cytometry and cumulative data is shown (Mean±SEM). The results in FIG. 5H to FIG. 5J indicate significant benefit of UCP2 inhibition combined with IL-17 blockade, while recapitulate no impact, or limited improvement with singular UCP2 or IL-17 targeting, respectively. The results in FIG. 5K to FIG. 5N indicate that UCP2 inhibition combined with IL-17 blockade increased the tumor-infiltrating populations of Tc1 cells, Th1 cells, and MDSC in PDAC-bearing mice. To further the translational value of this combinatorial immunotherapy approach, UCP2 inhibitor with a recombinant anti-IL-17 receptor antibody (αIL-17RA) were combined to examine the PDAC-directed antitumor efficacy thereof.

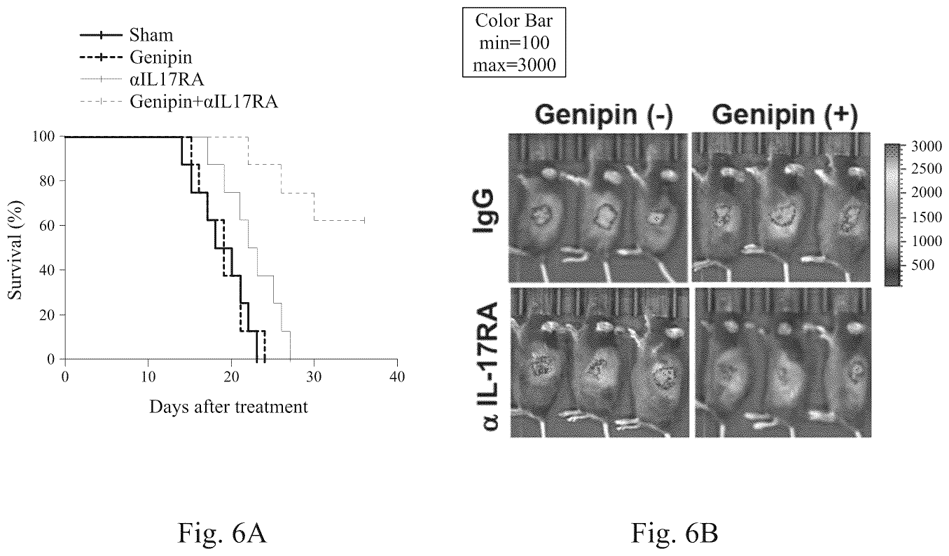

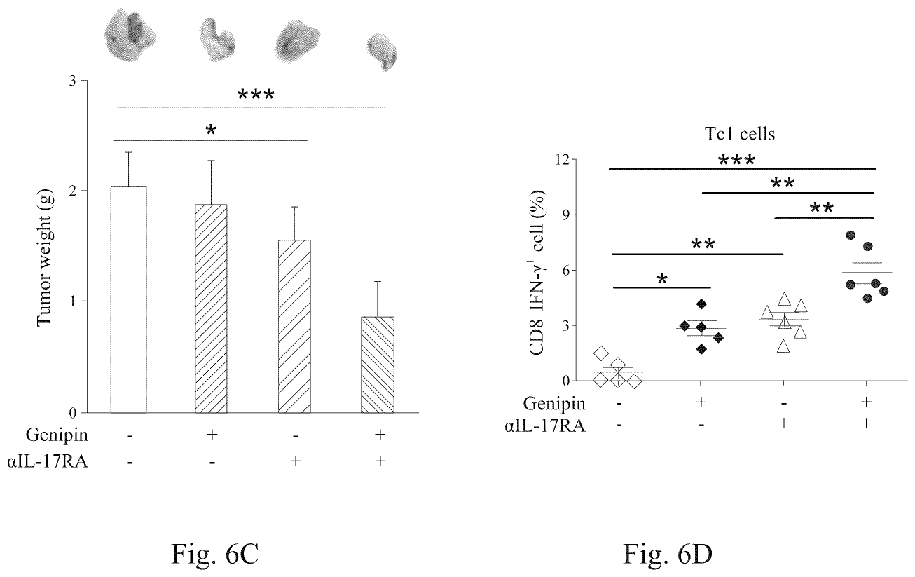

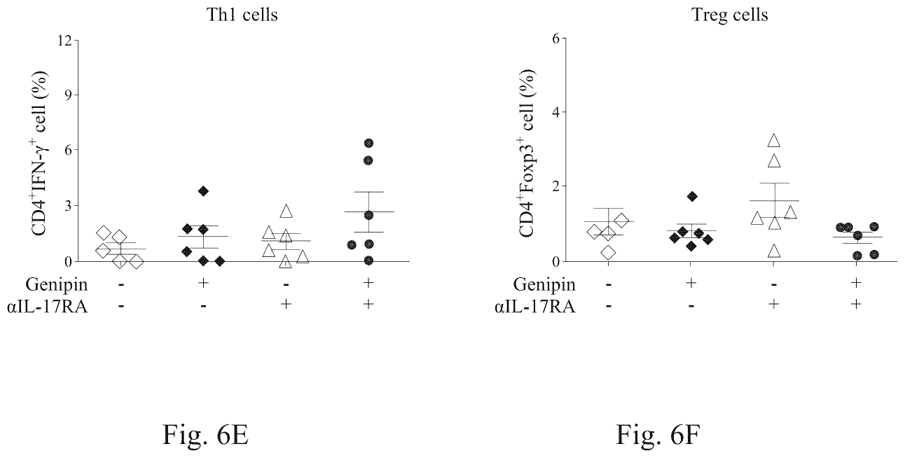

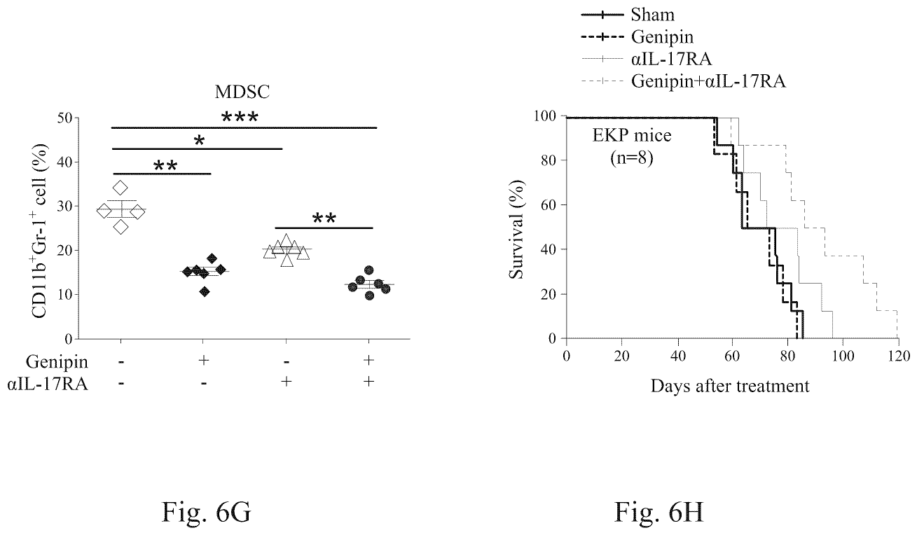

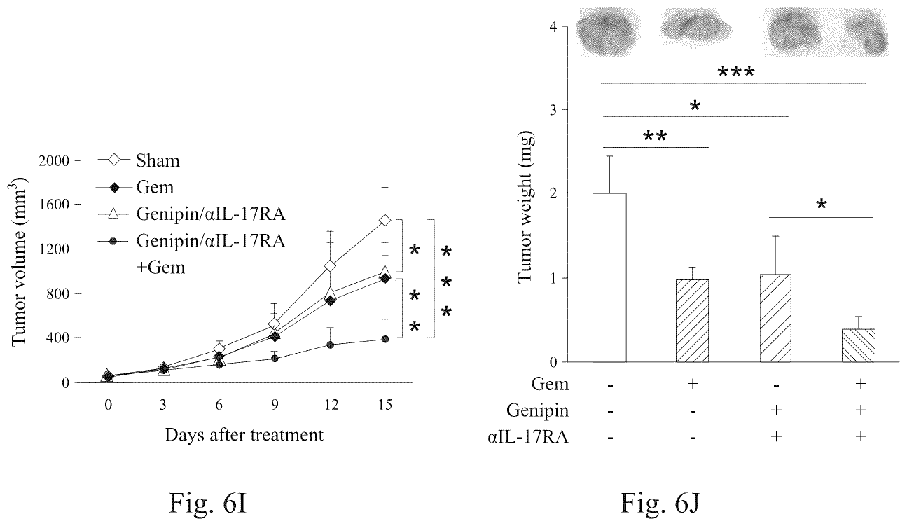

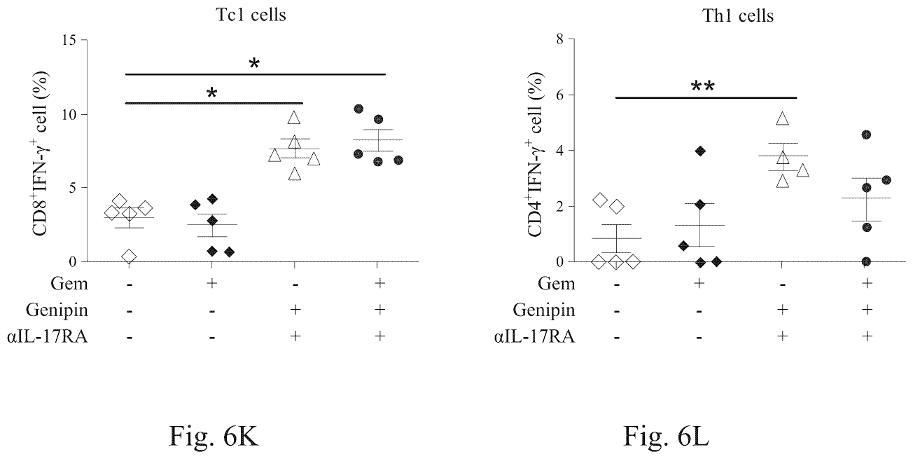

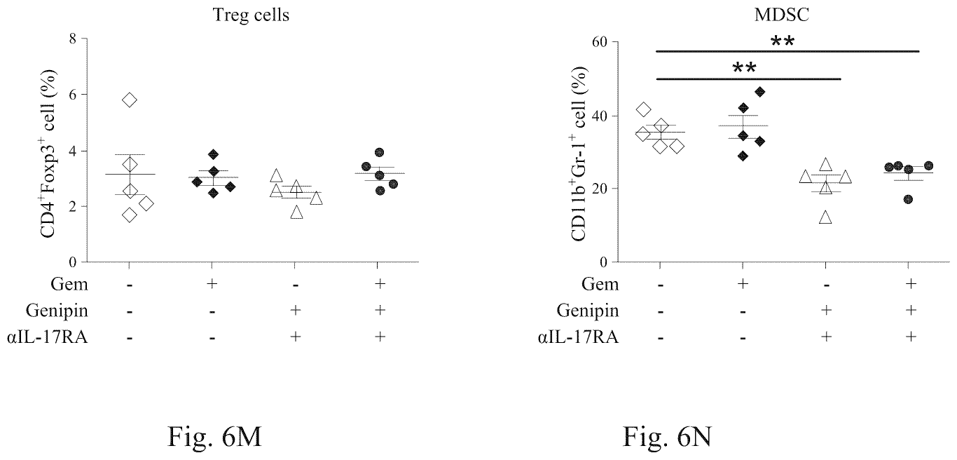

Reference is made to FIG. 6A to FIG. 6C, which show the results of survival, bioluminescence monitoring, and tumor weight analysis of orthotopic PDAC-bearing WT mice treated with genipin and αIL-17RA. WT mice underwent orthotopic implantation of Pan18 cells (N=6), followed by treatment with genipin (10 mg/kg, i.p., every two days) and αIL-17RA (1 mg/kg, i.v., once a week) for 3 weeks. Overall survival (N=8) in FIG. 6A, systemic tumor burden (N=5-6) in FIG. 6B, and tumor weight (N=5-6) in FIG. 6C were analyzed. Reference is made to FIG. 6D to FIG. 6G, which show the results of tumor immunity in PDAC-bearing WT mice treated with combination of genipin and αIL-17RA (N=5-6). Populations of Tc1 cells in PDAC-bearing mice in FIG. 6D, populations of Th1 cells in PDAC-bearing mice in FIG. 6E, populations of Treg cells in PDAC-bearing mice in FIG. 6F, and populations of MDSC in PDAC-bearing mice in FIG. 6G were assessed by flow cytometry and cumulative data is shown. Benefits of the combined treatment which substantially prolonged survival and decreased tumor growth were consistently observed (FIG. 6A to FIG. 6C). In addition, the combined treatment group demonstrated increased tumor-infiltration by Tc1 cells and reduced numbers of MDSC when compared to the groups treated with genipin or αIL-17RA alone (FIG. 6D to FIG. 6G).

Reference is made to FIG. 6H, which shows survival of PDAC-bearing transgenic EKP mice (represented as EKP mice) treated with genipin and αIL-17RA. PDAC-bearing transgenic EKP mice were treated with genipin and αIL-17RA per described schedule until the end of the experiment (N=8). To further the observations and recapitulate de novo progression of pancreatic cancer, the model of inducible PDAC generated in pancreas-specific KrasG12D knockin mice (LSL-Kras^{G12D/+}; p53^{+/-}; Ela-CreERT; EKP mice) upon cerulein treatment was employed, yet again documenting marked prolongation of survival in tumor bearing mice upon combined the UCP2 inhibitor (genipin) and the IL-17 blockade (αIL-17RA) use.

Reference is made to FIG. 6I to FIG. 6N, which show that tumorigenesis of orthotopic PDAC-bearing mice treated with combination of genipin, αIL-17RA, and gemcitabine (Gem). Genipin and αIL-17RA were administered to orthotopic PDAC-bearing mice with or without gemcitabine (100 mg/kg, i.p., every 3 days) for 3 weeks (N=5). Tumor volume in FIG. 6I and tumor weight in FIG. 6J were analyzed. Populations of Tc1 cells in orthotopic PDAC-bearing mice in FIG. 6K, populations of Th1 cells in orthotopic PDAC-bearing mice in FIG. 6L, populations of Treg cells in orthotopic PDAC-bearing mice in FIG. 6M, and populations of MDSC in orthotopic PDAC-bearing mice in FIG. 6N were assessed by flow cytometry and cumulative data is shown. When the UCP2 inhibitor combined with and the IL-17 blockade were integrated with gemcitabine, a standard chemotherapeutic agent used in advanced PDAC, a substantial enhancement of anti-tumor efficacy was observed (FIG. 6I and FIG. 6J), and cellular hallmarks of successful anti-tumor immunity was observed (FIG. 6K to FIG. 6N). In summary, combination of the UCP2 inhibitor and the IL-17 blockade demonstrates therapeutic efficacy in preclinical PDAC models with translational relevance and can be further optimized by combining the standard chemotherapy interventions to maximize therapeutic benefits.

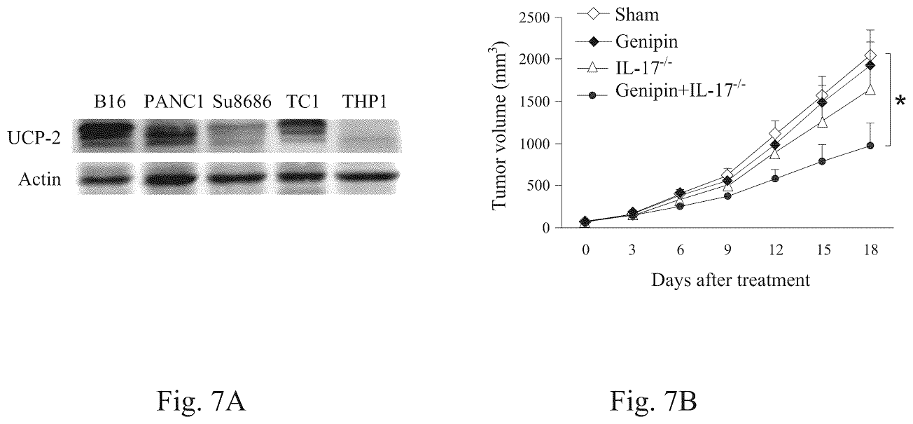

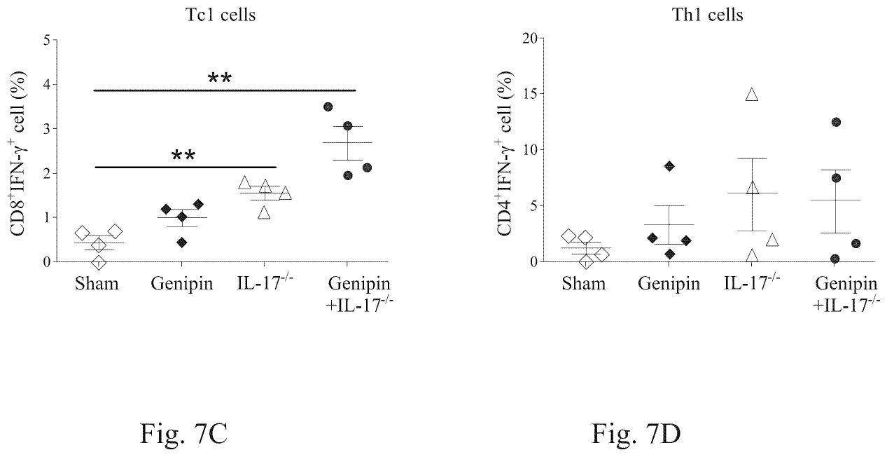

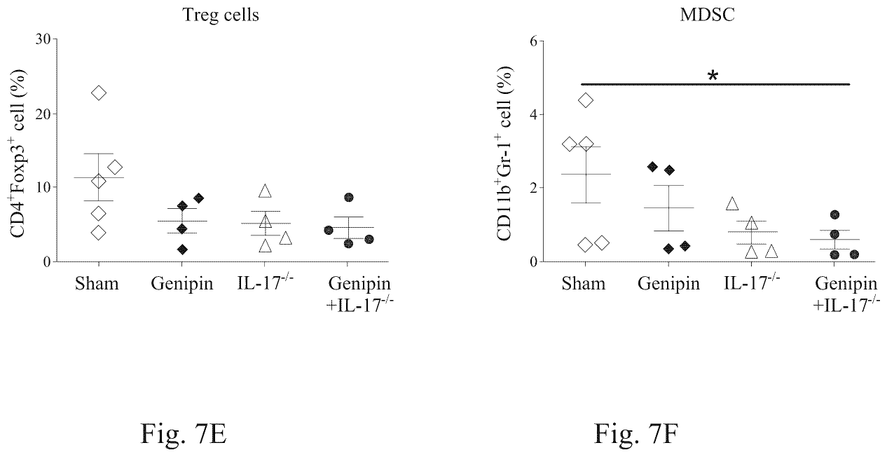

To test the broader anti-cancer efficacy of the therapeutic approach, the levels of UCP2 in different types of cancer cells were studied. Reference is made to FIG. 7A to FIG. 7F, which show that UCP2 inhibition combined with IL-17 depletion attenuates tumor growth and improves anti-tumor immunity in melanoma-bearing mice. FIG. 7A shows UCP2 expression in different types of cancer cell lines, wherein B16 is melanoma, PANC1 and Su8686 are pancreatic tumor, TC1 is myeloma, and THP1 is leukemia. FIG. 7B to FIG. 7F show the results of tumor volume and tumor immunity in genipin-treated melanoma-bearing IL-17-/- mice. Mice were subcutaneously implanted with B16F10 (2×10⁵ cells/mouse, N=4-5). Genipin (10 mg/kg, i.p., every two days) was administered to melanoma-bearing mice, including melanoma-bearing WT mice and melanoma-bearing IL-17/mice, for 3 weeks (N=4). Tumor volume in FIG. 7B was analyzed every two days. Populations of Tc1 cells in melanoma-bearing mice in FIG. 7C, populations of Th1 cells in melanoma-bearing mice in FIG. 7D, populations of Treg cells in melanoma-bearing mice in FIG. 7E, and populations of MDSC in melanoma-bearing mice in FIG. 7F were assessed by FACS analysis. The results in FIG. 7A show that relative abundance of UCP2 was evident in melanoma. The results in FIG. 7B show that the combination of the UCP2 inhibitor and the IL-17 blockade had an additional effect on the inhibition of tumor growth in melanoma-bearing mice. The results in FIG. 7C to FIG. 7E show that combined therapy increased the expansion of Tc1 cells while decreasing prevalence of MDSC in melanoma-bearing mice (FIG. 7C and FIG. 7F), while not significantly impacting populations of Th1 cells and Treg cells (FIG. 7D and FIG. 7E). Targeting UCP2 against tumorigenesis attributes to the induction of reactive oxygen species (ROS) and OXPHOS in melanoma cell. Herein, the pharmaceutical composition of the present disclosure including the UCP2 inhibitor and the IL-17 blockade attenuates tumorigenesis and improves tumor immunity in melanoma-bearing mice was proved.

5. UCP2 Depletion Enhances Anti-Tumor Immunity in Patients with PDAC

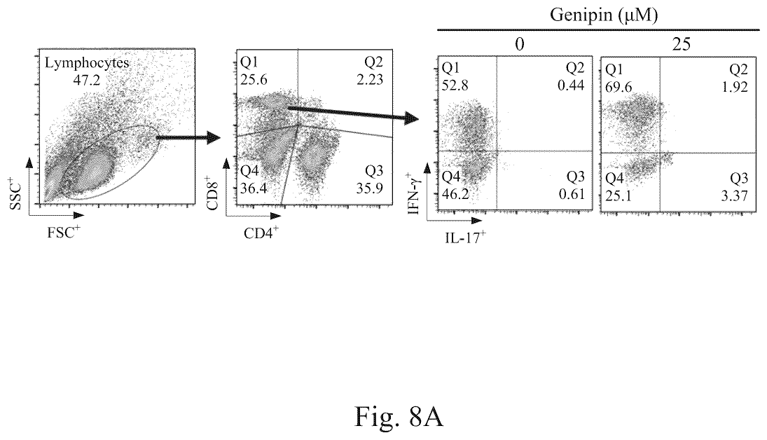

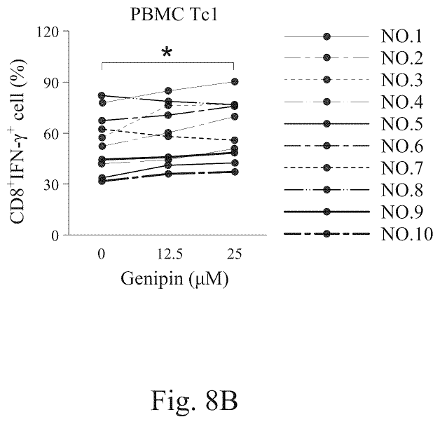

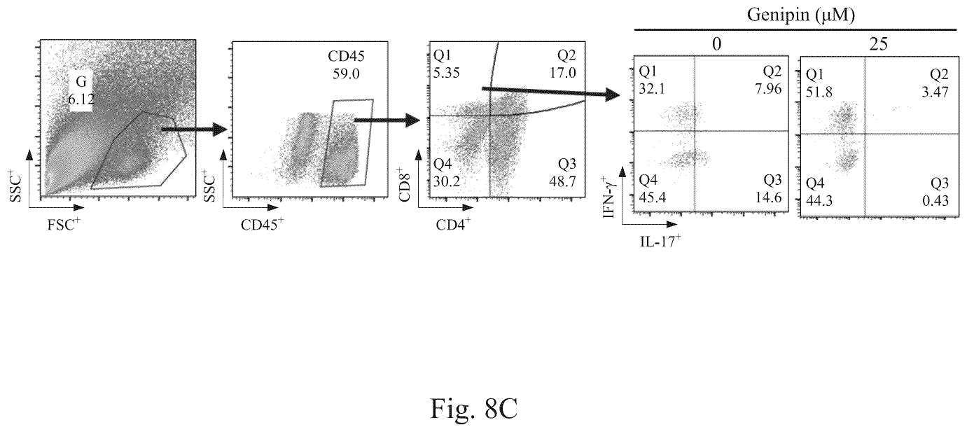

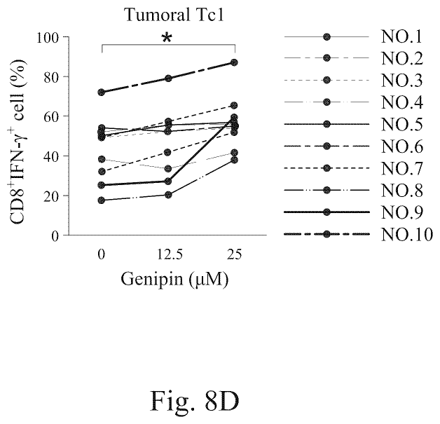

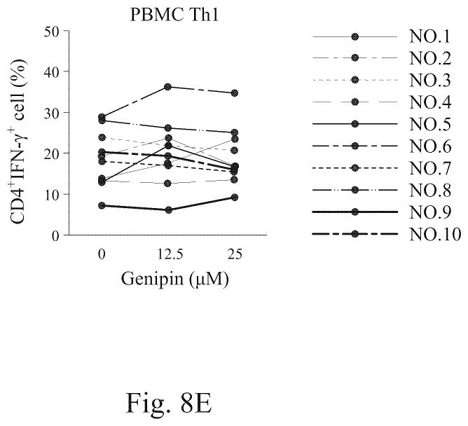

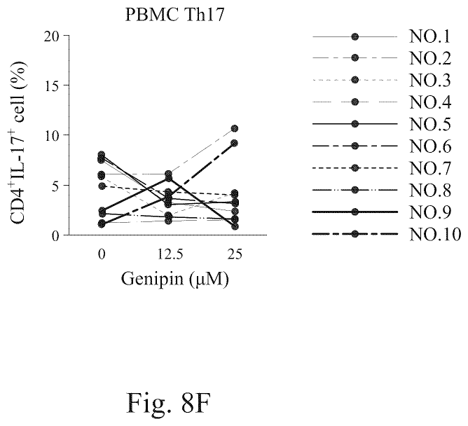

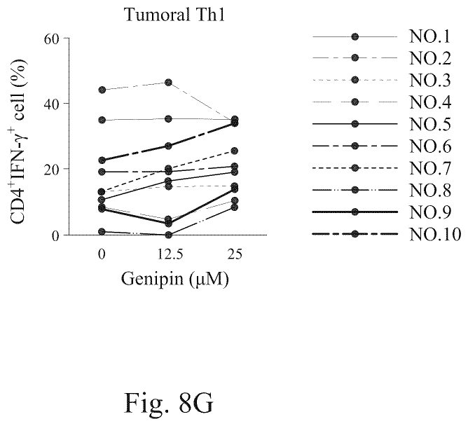

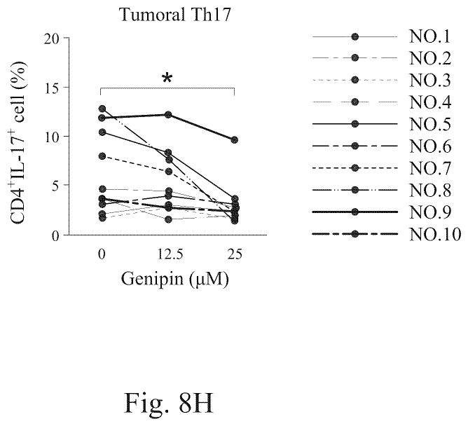

The anti-tumor immune effects of UCP2 inhibition in patients with PDAC were next investigated. Immune correlates of UCP2 inhibition in patient-derived PBMC and tumor samples, and flow cytometry was used to evaluate cytokine production of T cells after overnight-treatment with genipin. Reference is made to FIG. 8A to FIG. 8H, which show that UCP2 suppression facilitates IFN-γ production in peripheral blood and tumor infiltrating T cells retrieved from patients with PDAC. FIG. 8A and FIG. 8B show gating strategy, representative flow cytometry plots (FIG. 8A) and cumulative data (FIG. 8B) showing IFN-y-producing CD8⁺ T cells in genipin-treated PBMC of PDAC patients (N=10). FIG. 8C and FIG. 8D show gating strategy, representative flow cytometry plots (FIG. 8C) and cumulative data (FIG. 8D) showing IFN-γ-producing CD8⁺ T cells in genipin-treated tumors of PDAC patients (N=10). FIG. 8E and FIG. 8F show cumulative data showing IFN-γ-producing CD4⁺ T cells (FIG. 8E) and IL-17-producing CD4⁺ T cells (FIG. 8F) in genipin-treated PBMC of PDAC patients (N=10). FIG. 8G and FIG. 8H shows cumulative data showing IFN-γ-producing CD4⁺ T cells (FIG. 8G) and IL-17-producing CD4⁺ T cells (FIG. 8H) in genipin-treated tumors of PDAC patients (N=10).

The results show that UCP2 inhibition increased Tc1 cells not only in the PBMC (FIG. 8A and FIG. 8B), but in the tumors (FIG. 8C and FIG. 8D) of patients with PDAC. However, there were no significant changes on Th1 cells populations in the PBMC or tumors of patients (FIG. 8E to FIG. 8H). Interestingly, UCP2 inhibition did not increase PBMC Th17 cells or tumoral Th17 cells, which is a protumor cytokines in patients with PDAC (FIG. 8E to FIG. 8H). This inspiring result accord with the results of in vitro study (FIG. 1C and FIG. 1D) indicates the pharmaceutical composition of the present disclosure having the effect on the cancer immunotherapy against PDAC in patients.

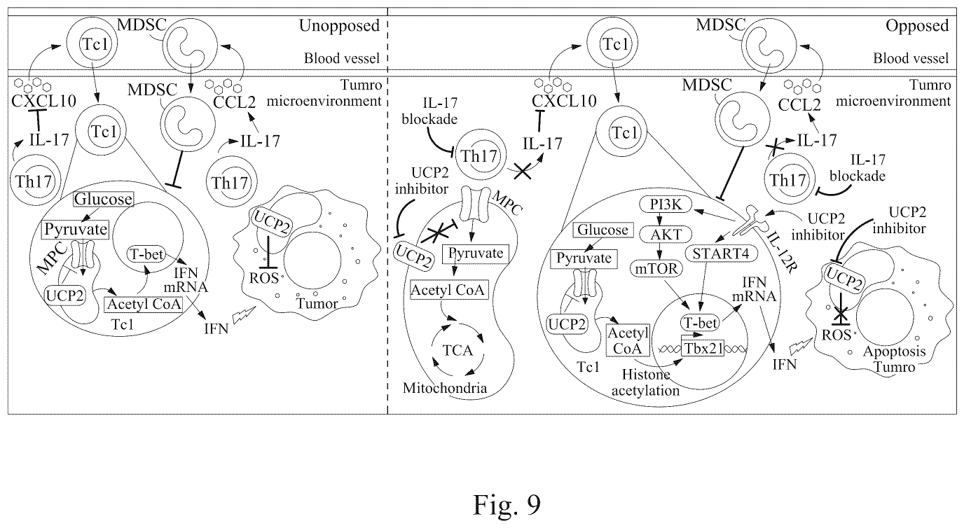

Reference is made to FIG. 9, which is a schematic view showing the pharmaceutical composition of the present disclosure enhancing antitumor immunity against PDAC. The present disclosure demonstrates that metabolic targeting of UCP2 offers promise of augmenting Tc1 cells (represented as Tc1) responses. This is achieved via upregulation of IL-12R/STAT4/mTOR signaling axis and consequent T-bet-mediated enhancement of IFN-γ production in Tc1 cells effectors. In vivo, UCP2 deficiency enhanced antitumor immunity and reduced PDAC-mediated immunosuppression in murine models. These benefits were further augmented by therapeutic IL-17 blockade, which tilted the chemokine signaling to favor effective anti-tumor responses. This included enhanced chemokine signals promoting tumor infiltration by Tc1 cells effectors while dampening MDSC-related immunosuppression. Observed therapeutic synergy provided tumor growth control in multiple preclinical models, including xenograft studies and offers a novel approach to address immune dysregulation associated with progressive malignancy.

To sum up, the efficacy of the pharmaceutical composition of the present disclosure including the UCP2 inhibitor and the IL-17 blockade on promoting the cancer immunotherapy is highlighted. The pharmaceutical composition of the present disclosure attenuates tumorigenesis and improves tumor immunity, thereby suppressing tumor growth in tumor-bearing mice. In addition, the pharmaceutical composition of the present disclosure not only promises immunostimulatory activity in ex vivo modeling but also furthers the efficacy when combined with the chemotherapeutic agent. The pharmaceutical composition of the present disclosure can target distinct immune functions offering augmented efficacy, limited toxicities, and lessen risk of resistance development, thereby enhancing limited the success of the cancer immunotherapy seen thus far. Therefore, the pharmaceutical composition of the present disclosure can be used for promoting the cancer immunotherapy to the subject in need thereof.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A pharmaceutical composition for promoting a cancer immunotherapy, comprising an uncoupling protein 2 (UCP2) inhibitor and an interleukin-17 (IL-17) blockade.

2. The pharmaceutical composition of claim 1, wherein the UCP2 inhibitor is a genipin, a Gardenia jasminoides Ellis or a siRNA targeting to an UCP2 gene.

3. The pharmaceutical composition of claim 1, wherein the IL-17 blockade is a monoclonal antibody targeting to IL-17, a recombinant anti-IL-17 receptor antibody (αIL-17RA) or a siRNA targeting to an IL-17A gene.

4. The pharmaceutical composition of claim 3, wherein the monoclonal antibody targeting to IL-17 is Secukinumab, ixekizumab or brodalumab.

5. The pharmaceutical composition of claim 1, further comprising a chemotherapeutic agent.

6. The pharmaceutical composition of claim 5, wherein the chemotherapeutic agent is gemcitabine.

7. The pharmaceutical composition of claim 1, wherein the cancer immunotherapy is for treating a pancreatic ductal adenocarcinoma or a melanoma.

8. A method for promoting a cancer immunotherapy comprising administering an effective concentration of a pharmaceutical composition to a subject in need thereof, wherein the pharmaceutical composition comprises an uncoupling protein 2 (UCP2) inhibitor and an interleukin-17 (IL-17) blockade.

9. The method of claim 8, wherein the UCP2 inhibitor is a genipin, a Gardenia jasminoides Ellis or a siRNA targeting to an UCP2 gene.

10. The method of claim 8, wherein the IL-17 blockade is a monoclonal antibody targeting to IL-17, a recombinant anti-IL-17 receptor antibody (αIL-17RA) or a siRNA targeting to an IL-17A gene.

11. The method of claim 10, wherein monoclonal antibody targeting to IL-17 is Secukinumab, ixekizumab or brodalumab.

12. The method of claim 8, the cancer immunotherapy is for treating a pancreatic ductal adenocarcinoma or a melanoma.
